(19) United States
(12) Patent Application Publication
(10) Pub. No.: US 2022/0387398 A1
(43) Pub. Date: Dec. 8, 2022

(54) IMMUNE TOLERIZATION AGENTS AND POTENTIATORS

(71) Applicant: AnTolRx, Inc., Cambridge, MA (US)

(72) Inventor: Vincent P. STANTON, Jr., Belmont, MA (US)

(21) Appl. No.: 17/626,540

(22) PCT Filed: Jul. 16, 2020
(86) PCT No.: PCT/US2020/042385

§ 371 Date: Jan. 12, 2022

Related U.S. Application Data
(60) Provisional application No. 62/874,795, filed on Jul. 16, 2019.

(51) Int. Cl. A61K 31/427; A61K 31/27; A61K 31/12; A61K 31/473; A61K 31/688; A61K 39/00; A61P 17/06; A61K 9/127

(52) U.S. Cl. CPC A61K 31/427; A61K 31/27; A61K 31/12; A61K 31/473; A61K 31/688; A61K 39/001; A61P 17/06; A61K 9/127; A61K 2039/5256

(57) ABSTRACT

The present invention features agents for treating autoimmune diseases and other conditions characterized by pathological immune responses, and for preventing or reversing immune recognition of neoantigens associated with therapeutic proteins or gene therapy vectors.

### FIELD OF THE INVENTION

The invention features tolerogenic nanoparticles for treating diseases and conditions associated with pathologic immune responses and methods for formulating and administering such compositions.

### BACKGROUND

The human immune system evolved to protect against external threats, including viruses, bacteria and parasites, yet these microorganisms are composed of the same types of molecules—proteins, lipids, carbohydrates, nucleic acids—as humans; indeed we share a common evolutionary history and some microbial macromolecules (e.g. proteins) share considerable homology with human counterparts. This overlap creates the potential for cross-reactivity between foreign and self-antigens. Immune system development therefore includes elaborate processes, referred to as tolerization, for eliminating self-reactive T and B cells. Tolerance for a specific antigen means that the host's immune system tolerates (does not mount an immune response against) the antigen. Tolerance for self-antigens is the normal, healthy state.

Loss of tolerance to one or more self-antigens can result in an autoimmune disease, a diverse collection of conditions which can affect one or many organs (e.g., brain, peripheral nervous system, liver, kidney, pancreas, gastrointestinal tract, joints, skin, eye, and ear). Known autoimmune diseases include alopecia areata, autoimmune uveitis, multiple sclerosis, psoriasis, rheumatoid arthritis, scleroderma, systemic lupus erythematosus, type 1 diabetes, vitiligo, and many others.

Restoration of tolerance in autoimmune diseases is a long-sought, but so far elusive therapeutic goal. It was demonstrated over three decades ago that administration of disease-associated auto-antigens in a variety of formulations and routes of administration can be effective at suppressing disease in animal models of autoimmune disease (e.g., experimental autoimmune encephalomyelitis in mice and rats and the non-obese diabetic (NOD) mouse model of autoimmune diabetes), but translation of these approaches to human patients has revealed various challenges.

Delivery of a tolerogenic signal to immune cells, or co-delivery of antigen and a tolerogenic signal, are promising approaches, supported by animal data, but they have not yet been adequately tested in humans to draw conclusions about their potential utility.

Existing therapies for autoimmune diseases either globally suppress immunity (e.g., anti-proliferative agents like methotrexate, azathioprine or leflunomide), target one arm of the immune system (e.g., the B-cell depleting antibody, rituximab), or globally suppress proinflammatory signals (e.g., anti-tumor necrosis factor alpha (TNFα) antibodies like infliximab and adalimumab). Thus, there is a need in the field for improved therapeutic strategies for selectively restoring tolerance to one or more autoantigens in cases of pathogenic immunity, such as autoimmune disease, transplant rejection, and graft-versus host disease.

A variety of innovative medical treatments, including peptide and protein therapeutics and gene therapies (often delivered using viral vectors), entail exposing a patient's immune system to new antigens (neoantigens), which are often recognized as foreign. The ensuing immune response limits or blocks the effectiveness of these treatments, and may include dangerous side effects like anaphylaxis. There is a need for improved methods and compositions to prevent immune recognition of neoantigens.

### SUMMARY OF THE INVENTION

The present invention features agents for treating autoimmune diseases and other conditions characterized by pathological immune responses, and for preventing or reversing immune recognition of neoantigens associated with therapeutic proteins or gene therapy vectors, including combinations of small molecules with complementary pharmacological activities (e.g. agent A induces tolerance, agent B extends the life of agent A), optionally delivered together with pathogenic auto-antigens to antigen presenting cells and/or T cells; formulation methods, including nanoparticulate formulations for preferentially delivering said agents to targeted cell populations (e.g., antigen presenting cells and/or T cells); delivery methods, including methods for oral, parenteral and dermal administration; and methods for treating autoimmune diseases. In one aspect, the invention features a composition comprising 2-(1 H-Indol-3-ylcarbonyl)-4-thiazolecarboxylic acid methyl ester (ITE), or salt thereof, and an inhibitor of ITE degradation. In some embodiments, the composition comprises a population of nanoparticles, wherein the population of nanoparticles comprises the ITE, or salt thereof. In some embodiments, the inhibitor of ITE degradation is an esterase inhibitor. Esterase inhibitors useful in the present invention include esterase inhibitors that inhibits multiple esterases. For example, the esterase inhibitor may inhibit a carboxylesterase, a plasma esterase, an acetylcholinesterase, a butyrylcholinesterase, and/or a paraoxonase. In some embodiments, the esterase inhibitor inhibits at least two, or at least three, esterase inhibitors from the group consisting of carboxylesterase 1, carboxylesterase 2, acetylcholinesterase, butyrylcholinesterase, or paraoxonase, e.g., at pharmacologically achievable concentrations. In some embodiments, the esterase inhibitor is selected from the group consisting of rivastigmine, benzil, tacrine, and bis-para-nitrophenylphosphate (BNPP).

In some embodiments, the ITE and the inhibitor of ITE are co-formulated. In other embodiments, the ITE and the inhibitor of ITE are separately formulated (e.g., for administration at separate times, frequencies, or by separate routes).

In some embodiments, the population of nanoparticles is a population of liposomes. In some embodiments, the population of nanoparticles (e.g., nanoparticulate liposomes) has an average diameter from 50-250 nanometers (nm; e.g., from 50 to 250 nm, from 50 to 200 nm, from 50 to 150 nm, from 75 to 125 nm, from 80 to 120 nm, from 90 to 110 nm, or from 95 to 105 nm, e.g., about 60 nm, about 70 nm, about 80 nm, about 90 nm, about 95 nm, about 100 nm, about 105 nm, about 110 nm, about 120 nm, or about 125 nm). In some embodiments, the population of nanoparticles is a population of nanoparticulate liposomes with an average desaturation index of 0.4 or greater (e.g., from 0.4-1.0, from 0.5-0.9, or from 0.6-0.8, e.g., from 0.4-0.5, from 0.5-0.6, from 0.6-0.7, from 0.7-0.8, from 0.8-0.9, or from 0.9-1.0). In some embodiments, the population of nanoparticles (e.g., nanoparticulate liposomes) has an average from 200-15,000 molecules of 2-(1H-Indol)-3-ylcarbonyl)-4-thiazolecarboxylic acid methyl ester (ITE), or salt thereof, per nanoparticle. In some embodiments, the population of nanoparticles is a population of nanoparticulate liposomes having an average phase transition temperature from −60° C. to 80° C. In some embodiments, the population of nanoparticles is a population of nanoparticulate liposomes having a lipid mixture comprising a saturated lipid species and an unsaturated lipid species, wherein the unsaturated lipid species comprises an unsaturated bond and accounts for at least 20% of the lipid mixture (e.g., at least 20%, at least 25%, at least 30%, at least 35%, at least 40%, at least 45%, at least 50% or more, by mass or by molar percentage). In some embodiments, the unsaturated lipid species has two lipid tails, wherein one or both lipid tails includes a single unsaturated bond.

In some embodiments, the population of nanoparticles (e.g., nanoparticulate liposomes) further has an antigen (e.g., a peptide antigen) at a mass ratio of antigen-to-ITE from 1:10 to 10:1 (e.g., from 1:100-10:1). The antigen can be a peptide antigen and/or an antigen is associated with an autoimmune disorder. In some embodiments, the autoimmune disorder is an autoimmune skin disease. For example, an antigen associated with pemphigus vulgaris can be desmoglein 3. In some embodiments, the autoimmune skin disease is psoriasis, alopecia areata, bullous pemphigoid, dermatomyositis, epidermolysis bullosa, pemphigus vulgaris, scleroderma, or vitiligo. In other embodiments, the autoimmune disorder is rheumatoid arthritis, multiple sclerosis, type I diabetes, myasthenia gravis, inflammatory bowel disease, or celiac disease. In some embodiments, the invention provides methods of tolerizing a subject to a therapeutic agent. In some embodiments, the invention provides methods of reducing immune response (e.g., cytokine secretion, antibody generation etc.) directed to a therapeutic agent. In some embodiments, the antigen is a therapeutic agent, e.g., a therapeutic protein or a portion thereof. In some embodiments, the antigen is derived from a vector used to deliver a nucleic acid (i.e., a gene therapy vector). Gene therapy vectors include natural and engineered viral vectors as well as non-viral proteins, glycoproteins, lipids, polysaccharides and other natural or non-natural polymers used to package and protect nucleic acids from nucleases, and/or to target nucleic acids to specific organs or cell types. The nucleic acid may encode a gene or part of a gene, or it may comprise DNA fragments used to correct an endogenous gene, for example, as in CRISPR (clustered regularly interspaced short palindromic repeats) gene editing technology. Any component of a gene therapy vector which can be recognized by the immune system (i.e., any neoantigen) constitutes an antigen against which induction of immune tolerance may improve the utility of gene therapy.

In some embodiments, the therapeutic agent is immunogenic. In some embodiments, the therapeutic agent is a therapeutic protein or peptide. In some embodiments, the therapeutic agent is a virus or capsid protein thereof. In other embodiments, the therapeutic agent is a polynucleotide encoding the therapeutic protein or peptide and/or the virus or capsid protein thereof.

In some embodiments, the therapeutic agent is a viral vector, e.g., an AAV, or capsid protein thereof. In some embodiments, the viral vector, e.g., AAV, includes DNA. In some embodiments, the DNA is a single stranded DNA (ssDNA), such as, a cDNA or fragment thereof (e.g., a human cDNA or fragment thereof). In other embodiments, the viral vector, e.g., AAV includes RNA. In some embodiments, the RNA is a microRNA (miRNA).

In some embodiments, the population of nanoparticles (e.g., nanoparticulate liposomes) has an average zeta potential from −10 and −50 mv (e.g., from −15 mv to −45 mv, from −20 mv to −40 mv, or from −25 mv to −35 mv, e.g., from −10 mv to −15 mv, from −15 mv to −20 mv, from −20 mv to −25 mv, from −25 mv to -30 mv, from −30 mv to −35 mv, from −35 mv to −40 mv, from −40 mv to −45 mv, or from −45 mv to −50 my, e.g., about −10 mv, about −15 mv, about −20 mv, about −25 mv, about −30 mv, about −35 mv, about −40 my, about −45 mv, or about −50 mv).

In another aspect, the invention features a method of treating a subject having an autoimmune disorder, the method comprising administering to the subject any of the compositions described herein in a therapeutically effective amount. In some embodiments, the administration is an intravenous, subcutaneous, intradermal, dermal, intra-articular, pulmonary, or mucosal administration. In some embodiments, the amount of ITE in a single dose of nanoparticles is between about 1 μg and 10 mg (e.g., from 10 μg to 10 mg, from 50 μg to 5 mg, from 100 μg to 1 mg, from 150 μg to 500 μg, from 200 μg to 400 μg, or from 250 μg to 250 μg, e.g., from 1 μg to 10 μg, from 10 μg to 50 μg, from 50 μg to 100 μg, from 100 μg to 150 μg, from 150 μg to 200 μg, from 200 μg to 250 μg, from 250 μg to 300 μg, from 300 μg to 350 μg, form 350 μg to 400 μg, from 400 μg to 450 μg, from 450 μg to 500 μg, from 500 μg to 600 μg, from 600 μg to 700 μg, from 700 μg to 800 μg, from 800 μg to 900 μg, from 900 μg to 1.0 mg, from 1.0 mg to 5.0 mg, or from 5.0 mg to 10 mg). For example, the amount of ITE in a single dose of liposomes can be from 100 μg to 500 μg (e.g., from 200 μg to 300 μg, e.g., about 200 μg, about 210 μg, about 220 μg, about 230 μg, about 240 μg, about 250 μg, about 260 μg, about 270 μg, about 280 μg, about 290 μg, or about 300 μg).

In some embodiments, the amount of inhibitor of ITE in a single dose of nanoparticles is between about 1 μg and 50 mg (e.g., from 10 μg to 50 mg, from 50 μg to 20 mg, from 100 μg to 10 mg, from 150 μg to 1 mg, from 200 μg to 500 μg, or from 250 μg to 250 μg, e.g., from 1 μg to 10 μg, from 10 μg to 50 μg, from 50 μg to 100 μg, from 100 μg to 150 μg, from 150 μg to 200 μg, from 200 μg to 250 μg, from 250 μg to 300 μg, from 300 μg to 350 μg, form 350 μg to 400 μg, from 400 μg to 450 μg, from 450 μg to 500 μg, from 500 μg to 600 μg, from 600 μg to 700 μg, from 700 μg to 800 μg, from 800 μg to 900 μg, from 900 μg to 1.0 mg, from 1.0 mg to 5.0 mg, from 5.0 mg to 10 mg, from 10 mg to 20 mg, from 20 mg to 30 mg, from 30 mg to 40 mg, or from 40 mg to 50 mg). In some embodiments, a dose of the composition administered comprises from 1 mg to 20 mg inhibitor of ITE degradation. In some embodiments, a dose of the composition administered comprises from 1 μg to 50 mg ITE and from 10 μg to 50 mg inhibitor of ITE degradation.

In another aspect, the invention features a method of treating a subject having an autoimmune disorder, the method comprising administering to the subject (a) a population of nanoparticles comprising ITE, or salt thereof (e.g., any of the populations of nanoparticles described herein), and (b) an inhibitor of ITE degradation (e.g., any of the ITE degradation inhibitors described herein). The population of nanoparticles may be administered through a separate route than the inhibitor of ITE degradation. In some embodiments, the inhibitor of ITE degradation is administered with an antigen, wherein the antigen is associated with the autoimmune disorder, as described herein. In some embodiments, the inhibitor of ITE degradation is formulated in a gel, lotion, or cream for topical administration. In some embodiments, the autoimmune disorder is an autoimmune skin disease, such as psoriasis, alopecia areata, bullous pemphigoid, dermatomyositis, epidermolysis bullosa, pemphigus vulgaris, scleroderma, vitiligo.

In another aspect, the invention features methods for improving the efficacy and safety of peptide, protein or gene therapy by inducing immune tolerance to one or more neoantigens associated with the therapeutic composition, and thereby improving the pharmacokinetics (e.g., prolonging the half-life), the delivery (e.g., increasing the efficiency of viral transduction), the efficacy of multiple dose treatment regimens (which would otherwise induce blocking immune responses) and the safety (e.g., preventing allergic reactions) of treatment, among other benefits. The methods may include administering to the subject the composition of any of the preceding embodiments, via the routes of administration of any of the preceding embodiments, in a therapeutically effective amount.

### BRIEF DESCRIPTION OF THE DRAWINGS

FIGS. 1A and 1B are graphs showing calibration curves for ITE (FIG. 1A) and ITC (FIG. 1 B).

FIG. 2A is a graph showing the rate of degradation of ITE in mouse plasma in the presence of rivastigmine (gray squares), benzyl (gray diamonds), BNPP (black triangles), tacrine (gray dots), cocktail (gray triangles), compared to the rate of degradation of ITE in mouse plasma in the absence of esterase inhibitor (dark gray circles; control).

FIG. 2B is a graph showing the rate of ITE (dark line) degradation, the rate of ITC (light line) appearance in human plasma over two hours, and the sum of ITE+ITC (medium line).

FIG. 3 is a graph showing the rate of ITE (dark line) degradation and the rate of ITC (light line) appearance in human plasma over two hours, and the sum of ITE+ITC (medium line) in the presence of benzil.

FIG. 4 is a graph showing the rate of ITE (dark line) degradation and the rate of ITC (light line) appearance in human plasma over two hours, and the sum of ITE+ITC (medium line) in the presence of tacrine.

FIG. 5 is a graph showing the rate of ITE (dark line) degradation and the rate of ITC (light line) appearance in human plasma over two hours, and the sum of ITE+ITC (medium line) in the presence of bis-para-nitrophenylphosphate (BNPP).

FIG. 6 is a graph showing the rate of ITE (dark line) degradation and the rate of ITC (light line) appearance in human plasma over two hours, and the sum of ITE+ITC (medium line) in the presence of rivastigmine.

FIG. 7 is a graph showing the rate of ITE (dark line) degradation and the rate of ITC (light line) appearance in human plasma over two hours, and the sum of ITE+ITC (medium line) in the presence of cocktail.

FIGS. 8A-8D is a series of graphs showing relative efficacy of ITE-loaded nanoparticles containing esterase inhibitor in treating psoriasis. FIG. 8A shows scaling scores; FIG. 8B shows thickness scores; FIG. 8C shows erythema scores; and FIG. 8D shows cumulative scores. Upward-facing triangles represent untreated, healthy controls (naïve); gray squares represent plain nanoparticulate liposomes (NLPs); gray circles represent ITE-NLPs; downward-facing triangles represent plain NLPs with rivastigmine; and gray diamonds represent ITE-NLPs with rivastigmine. * represents statistical significance between ITE-NLPs with rivastigmine vs. plain NLPs; δ represents statistical significance between ITE-NLPs with rivastigmine vs. plain NLPs with rivastigmine; and # represents statistical significance between ITE-NLPs with rivastigmine vs. ITE-NLPs without rivastigmine. One, two, and three symbols denote p<0.05, p<0.01, and p<0.001, respectively.

FIGS. 9A-9E is a series of photographs showing visual characterization of psoriasis symptoms. FIG. 9A shows an untreated, healthy control mouse (naïve); FIG. 9B shows a psoriasis mouse treated with plain NLPs; FIG. 9C shows a psoriasis mouse treated with ITE-NLPs; FIG. 9D shows a psoriasis mouse treated with plain NLPs with rivastigmine; and FIG. 9E shows a psoriasis mouse treated with ITE/NLPs with rivastigmine.

FIGS. 10A and 10B are a set of bar graphs showing reduced expression of inflammatory cytokines IL-17 (FIG. 10A) and IL-22 (FIG. 10B) in response to treatment with ITE-NLPs with rivastigmine.

### DETAILED DESCRIPTION

The present invention features tolerogenic nanoparticles (e.g., nanoparticulate liposomes) useful as treatment for diseases, such as autoimmune diseases (e.g., rheumatoid arthritis, multiple sclerosis, type I diabetes, myasthenia gravis, inflammatory bowel disease, and celiac disease). In particular, the invention features nanoparticles having a composition configured to deliver 2-(1 H-Indol-3-ylcarbonyl)-4-thiazolecarboxylic acid methyl ester (ITE), or salt thereof, together with an inhibitor of ITE degradation (e.g., an esterase inhibitor).

### Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “nanoparticle” refers to a synthetic particle having at least one dimension (e.g., diameter) of between 1 and 1,000 nanometers (nm). In particular embodiments, the nanoparticle is substantially spherical, and the diameter can be measured using any suitable method known in the art, such as dynamic light scattering (DLS; e.g., using a ZetaSizer instrument; Malvern, UK), Brownian motion analysis (e.g., nanoparticle tracking analysis, as provided by NanoSight instruments; Malvern, UK), etc. Nanoparticles include nanoparticulate liposomes, micelles, and other polymeric particles, such as poly-lactic-co-glycolic acid (PLGA) particles, which can be synthesized and characterized according to suitable methods known in the art.

As used herein, two agents are “co-formulated” by mixing the agents prior to, or during, formulation of the nanoparticles, such that both agents are present in an individual nanoparticle. Alternatively, two agents are separately formulated by generating two populations of nanoparticles, each of which includes only one of two agents. In some embodiments, ITE and an inhibitor of ITE are separately formulated and co-administered.

As used herein, the term “micelle” refers to a substantially spherical particle (e.g., a nanoparticle) characterized by a hydrophobic core and a hydrophilic shell formed by assembly of amphiphilic molecules (e.g., amphiphilic polymers). In certain embodiments, micelles self-assemble into the core-shell configuration when the amphiphilic polymer exceeds a critical threshold concentration in a suitable solvent (e.g., an aqueous solution). The exterior (hydrophilic) moiety may be a polyethylene glycol (e.g., PEG-500, PEG-1000, PEG-2000, PEG-3000, PEG-5000, where the number refers to the number of units of ethylene glycol). The interior moiety of the amphiphile can be a lipid, a polymer, an amino acid, an aromatic compound, or a combination. Examples of micelles include polymer-lipid micelles (e.g., with polyethylene glycol on the outside and a fatty acid, such as oleic acid, on the inside), polymer-phospholipid-micelles (e.g., hybrid polymeric micelles (e.g. with a hydrophilic polymer, such as polyethylene glycol on the outside and a relatively hydrophobic polymer, such as poly(D,L-lactide) in the interior). The hydrophilic and hydrophobic moieties of the amphiphile can be joined by a variety of chemical groups using standard attachment chemistry. For example, PEG may be joined to a lipid via phosphoglycerol. Amide, ester, and other linkages are also known in the art. Amphiphiles suitable for forming micelles are commercially available from a variety of vendors (e.g. Avanti Polar Lipids, Millipore-Sigma).

As used herein, the term “desaturation index” refers to the weighted average of the number of double bonds per fatty acid. For example, a liposome having an equimolar amount of two lipid species, wherein one species has two saturated fatty acid tails, and the other species has one saturated tail and one monounsaturated tail, the liposome has a desaturation index of 0.25 (i.e., 1 in 4 fatty acid tails are unsaturated).

As used herein, the term “subject” includes any mammal in need of the methods of treatment or prophylaxis described herein. In some embodiments, the subject is a human. Other mammals in need of such treatment or prophylaxis include dogs, cats, or other domesticated animals, horses, livestock, laboratory animals, including non-human primates, etc. The subject may be male or female. In one embodiment, the subject has an autoimmune disease. In other embodiments, the subject is to receive peptide, protein, or gene therapy. The subject may be any age during which treatment or prophylactic therapy may be beneficial. For example, in some embodiments, the subject is 0-5 years of age, 5-10 years of age, 10-20 years of age, 20-30 years of age, 30-50 years of age, 50-70 years of age, or more than 70 years of age.

As used herein, the term “neoantigen” refers to a molecule or portion of a molecule associated with a therapeutic agent (e.g. peptide, protein or gene therapy) that is recognized by the immune system and is capable of inducing an immune response. The nature of the immune response may be humoral (e.g., neutralizing antibodies against the neoantigen that inhibit function, shorten half-life or trigger degradation), cellular (e.g., T cell receptor recognition of peptides derived from the neoantigen), or both. Immune responses to neoantigens generally interfere with therapeutic efficacy.

As used herein, the term “gene therapy” refers to any nucleic acid-based therapeutic (whether composed of natural nucleotides or non-natural nucleotides) designed to replace, correct (e.g., by gene editing or targeted recombination) or alter the expression of (e.g., by oligonucleotide-induced exon skipping or RNA interference) an endogenous gene, or, in the case of cancer therapy, provide a novel function designed to exploit a vulnerability of cancer cells.

As used herein, the term “gene therapy vector” refers to components of a gene therapy composition designed to protect (e.g., from nucleases), steer (e.g., to targeted cells or tissues), enhance cell penetrance of (e.g., by viral transduction), or otherwise improve the efficacy of gene therapy. Often one or more components of a gene therapy vector comprise non-native elements capable of inducing an immune response. Viral gene therapy vectors comprise one important category of gene therapy vectors. The most widely used viral vectors include adenoviruses, adeno-associated viruses (AAV), herpes simplex viruses, lentiviruses, poxviruses, retroviruses and vaccinia viruses, all of which have multiple variants. As used herein, an “effective amount” or “effective dose” of a composition thereof refers to an amount sufficient to achieve a desired biological (e.g., immunological) and/or pharmacological effect, e.g., when delivered to a cell or organism according to a selected administration form, route, and/or schedule. As will be appreciated by those of ordinary skill in this art, the absolute amount of a particular composition that is effective can vary depending on such factors as the desired biological or pharmacological endpoint (e.g., improved therapeutic efficacy or prolongation of therapeutic effect), the agent to be delivered, the target tissue, etc. Those of ordinary skill in the art will further understand that an “effective amount” can be contacted with cells or administered to a subject in a single dose or through use of multiple doses.

As used herein, the term “treatment,” or a grammatical derivation thereof, is defined as reducing the progression of a disease, reducing the severity of a disease symptom, retarding progression of a disease symptom, removing a disease symptom, or delaying onset of a disease.

As used herein, the term “prevention” of a disorder, or a grammatical derivation thereof, is defined as reducing the risk of onset of a disease, e.g., as a prophylactic therapy for a subject who is at risk of developing a disorder, e.g., an autoimmune disorder. A subject can be characterized as “at risk” of developing a disorder by identifying a mutation (e.g., a DNA sequence variation) associated with the disorder, according to any suitable method known in the art or described herein. Alternatively, a subject can be characterized as “at risk” of developing a disorder if the subject is positive for an autoantibody associated with the development of the disorder. Additionally or alternatively, a subject can be characterized as “at risk” of developing a disorder if the subject has a family history of the disorder.

The term “pharmaceutically acceptable” means safe for administration to a mammal, such as a human. In some embodiments, a pharmaceutically acceptable composition is approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which a vector or composition of the invention is administered. Examples of suitable pharmaceutical carriers are described in “Remington's Pharmaceutical Sciences,” Mack Publishing Co., Easton, PA., 2^{nd} edition, 2005.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within ±10% variability from the reference value, unless otherwise specified.

For any conflict in definitions between various sources or references, the definition provided herein shall control.

### Compositions

The invention provides nanoparticles (e.g., nanoparticulate liposomes) containing ITE for tolerization of a subject's immune system. Compositions of the present invention further include inhibitors of ITE degradation, which can increase the effective half-life of the nanoparticulate ITE.

In some embodiments, the compositions of the present invention enable delivery of less total ITE to a subject relative to that which would be therapeutically effective as delivered by a different means. In some embodiments, the population of nanoparticles has an average of 100-20,000 molecules of ITE (e.g., from 200-15,000 molecules of ITE, from 300-12,000 molecules of ITE, from 400-10,000 molecules of ITE, from 500-8,000 molecules of ITE, from 750-5,000 molecules of ITE, or from 1,000-3,000 molecules of ITE, e.g., from 100-200 molecules of ITE, from 200-500 molecules of ITE, from 500-1,000 molecules of ITE, from 1,000-2,500 molecules of ITE, from 2,500-5,000 molecules of ITE, from 5,000-7,500 molecules of ITE, from 7,500-10,000 molecules of ITE, from 10,000-15,000 molecules of ITE, or from 15,000-20,000 molecules of ITE).

Nanoparticles of the invention can be nanoparticulate liposomes. In other embodiments, nanoparticles of the invention can solid nanoparticles. For example, nanoparticles carrying ITE and an inhibitor of ITE degradation can be polymeric nanoparticles (e.g., biodegradable polymeric nanoparticles, such as PLGA nanoparticles). In particular embodiments, a population of nanoparticles (e.g., nanoparticulate liposomes) has an average (e.g., mean or median) diameter from 50 to 500 nm (e.g., from 50 to 250 nm, from 50 to 200 nm, from 50 to 150 nm, from 75 to 125 nm, from 80 to 120 nm, from 90 to 110 nm, or from 95 to 105 nm, e.g., about 60 nm, about 70 nm, about 80 nm, about 90 nm, about 95 nm, about 100 nm, about 105 nm, about 110 nm, about 120 nm, or about 125 nm).

Tolerogenic nanoparticles (e.g., nanoparticulate liposomes) may have a surface charge (i.e., zeta potential) from −10 mv to −50 mv (e.g., from −15 mv to −45 mv, from −20 mv to −40 mv, or from −25 mv to −35 mv, e.g., from −10 mv to −15 mv, from −15 mv to −20 mv, from −20 mv to −25 mv, from −25 mv to −30 mv, from −30 mv to −35 mv, from −35 mv to −40 mv, from −40 mv to −45 mv, or from −45 mv to −50 mv, e.g., about −10 mv, about −15 mv, about −20 mv, about −25 mv, about −30 mv, about −35 mv, about −40 mv, about −45 mv, or about −50 mv).

### Inhibitors of ITE Ddegradation

The invention features nanoparticles having ITE and an inhibitor of ITE degradation, configured to increase the effects of ITE by reducing its degradation (e.g., upon administration to the subject). ITE can be degradated by various enzymes endogenous to a subject, such as esterases, which can be present in the target cell that has taken up the nanoparticles or, alternatively, in the subject's circulation (e.g., in the plasma).

In some embodiments the ITE degradation inhibitor is an esterase inhibitor (e.g., an inhibitor of a carboxylesterase and/or a plasma esterase, such as acetylcholinesterase, butyrylcholinesterase, paraoxonase). In certain embodiments, the ITE degradation inhibitor is an agent that inhibits multiple esterases, including carboxylesterases and one or more plasma esterases (e.g., acetylcholinesterase, butyrylcholinesterase, paraoxonases). In some embodiments, the ITE degradation inhibitor is either an FDA approved drug or a natural product not regulated by the FDA. In one specific embodiment, the ITE degradation inhibitor is the FDA approved drug, rivastigmine. In other embodiments, the ITE degradation inhibitor is benzil, tacrine, bis-para-nitrophenylphosphate (BNPP), donepezil, galantamine, icopezil, pyridostigmine, edrophonium, neostigmine, physostigmine, Huperzine A, phenserine, tacrine, and pharmaceutically acceptable salts thereof.

### Lipids

In embodiments involving nanoparticulate liposomes, compositions of the invention are characterized by lipid compositions that enable efficient loading and delivery of ITE (e.g., to dendritic cells of the immune system). Liposomes made of lipids having one or more unsaturated (e.g., monounsaturated) fatty acid tails (e.g., wherein one or both tails contain at least one carbon-carbon double bond) provide for greater concentrations of ITE within the lipid bilayer. Such lipids having saturated fatty acid tails include, for example, egg phosphocholine (egg PC), 1,2-dioleoyl-sn-glycero-3-phosphocholine (DOPC), 1,2-dioleoyl-sn-glycero-3-phospho-L-serine (DOPS), oleic acid, palmitoleic acid, and cis-vaccenic acid. Egg PC has a double bond on one of its two fatty acid tails, whereas DOPC, DOPS, oleic acid, palmitoleic acid, and cis-vaccenic acid each have one double bond on both of its two fatty acid tails. Representative structures of egg PC, DOPC, and DOPS are shown below.

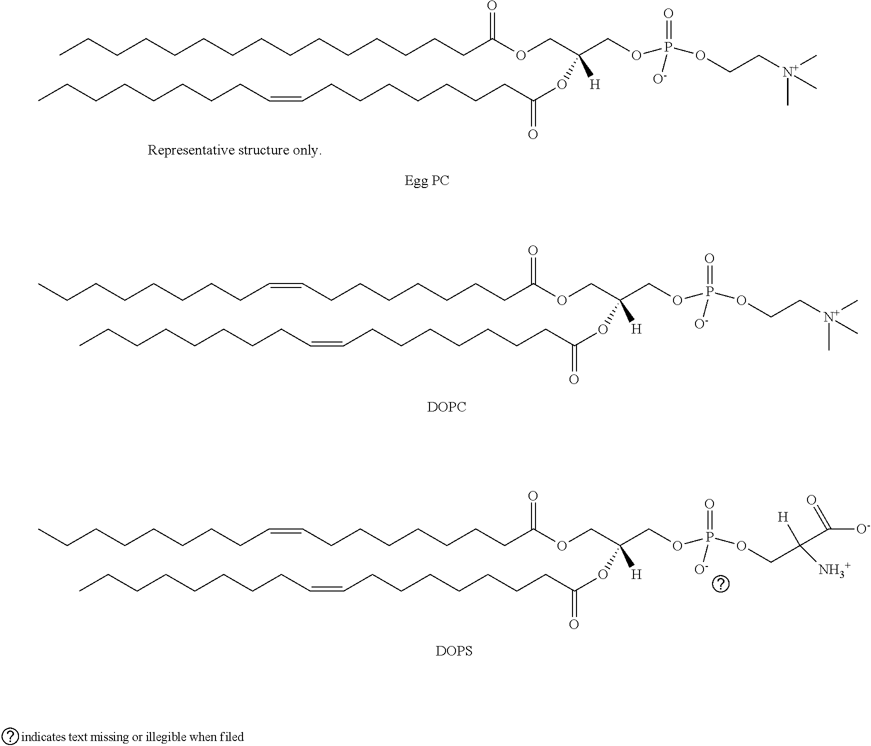

Lipids useful in the synthesis of ITE-loaded tolerogenic liposomes include those having fatty acid tails from 8-25 carbons in length (e.g., from 10-20, from 12-18, or from 14-16 carbons in length, e.g., from 8-10, 10-12, 12-14, 14-16, 16-18, 18-20, or 20-25 carbons in length, e.g., greater than 10, 11, 12, 13, 14, 15, 16, 17, 18, 19, 20, 21, 22, 23, 24, or 25 carbons in length). In some embodiments, lipids of the present liposomes having one or more (e.g., two) fatty acid tails greater than 14 carbons in length. In some embodiments, the lipid is unsaturated (e.g., it has at least one carbon-carbon double bond) in one or more (e.g., two) of its fatty acid tails. Such unsaturated lipids include egg PC, DOPC, and DOPS. A summary of monounsaturated fatty acids useful as part of the liposomes described herein are summarized below.

| | | |
|---|---|---|
| Common name | Structure | Chemical name |

Monounsaturated omega-7 fatty acids

| | | |
|---|---|---|
| None | 14:1 (n-7) | 7-Tetradecenoic acid |
| Palmitoleic acid | 16:1 (n-7) | 9-Hexadecenoic acid |
| Vaccenic acid | 18:1 (n-7) | 11-Octadecenoic acid |
| Paullinic acid | 20:1 (n-7) | 13-Eicosenoic acid |
| none | 22:1 (n-7) | 15-Docosenoic acid |

Monounsaturated omega-9 fatty acids

| | | |
|---|---|---|
| Oleic acid | 18:1 (n-9) | (Z)-octadec-9-enoic acid |
| Elaidic acid | 18:1 (n-9) | (E)-octadec-9-enoic acid |
| Gondoic acid | 20:1 (n-9) | (Z)-eicos-11-enoic acid |
| Erucic acid | 22:1 (n-9) | (Z)-docos-13-enoic acid |

Other lipids that may be useful for use as part of a liposome of the present invention include 1,2-dimyristoyl-sn-glycero-3-phosphocholine (DMPC); 1,2-distearoyl-sn-glycero-3-phosphocholine (DSPC); 1,2-distearoyl-sn-glycero-3-phosphoethanolamine-N-[methoxy(poly-ethylene glycol)-2000] (PEG2000-DSPE); and D-alpha-tocopherol poly(ethylene glycol)-1000 succinate (TPGS-1000).

The outward-facing phospholipid head group can affect interactions with immune cells. For example, phospholipids which terminate in L-serine (phosphatidylserine) can induce tolerance in immune cells. This property reflects the natural role of phospho-L-serine phospholipids in apoptosis. Ordinarily phosphatidylserine is located in the inner leaflet (layer) of the cell membrane. However, it appears in the outer leaflet of apoptotic cells or cell fragments, where it induces a tolerogenic response in phagocytic cells. Thus, in certain embodiments, phospholipids terminating in L-serine are useful, such as 1,2-dioleoyl-sn-glycero-3-phospho-L-serine (DOPS).

One or more unsaturated (e.g., monounsaturated) lipid species may account for at least 10% of the total lipids (on a molar basis, or by mass). In some embodiments, the unsaturated (e.g., monounsaturated) lipid species may account for at least 15%, at least 20%, at least 25%, at least 30%, at least 35%, at least 40%, at least 45%, at least 50%, at least 60%, at least 70%, at least 75%, at least 80%, at least 85%, or at least 90% of the total lipids of the liposome (e.g., from 10% to 20%, from 20% to 30%, from 30% to 40%, from 40% to 50%, from 50% to 60%, from 60% to 70%, from 70% to 80%, or from 80% to 90%, e.g., about 10%, about 15%, about 20%, about 25%, about 30%, about 35%, about 40%, about 45%, about 50%, about 55%, about 60%, about 65%, about 70%, about 75%, about 80%, about 85%, or about 90% of the total lipids of the liposome). In some embodiments, an unsaturated lipid species accounts for at least 25% of the total lipids.

In some embodiments, the population of liposomes has an average desaturation index of 0.3 or greater (e.g., at least 0.35, at least 0.4, at least 0.45, at least 0.5, at least 0.55, at least 0.6, at least 0.65, at least 0.7, at least 0.75, at least 0.8, at least 0.85, at least 0.9, e.g., from 0.4 to 0.5, from 0.5 to 0.6, from 0.6 to 0.7, from 0.7 to 0.8, or from 0.8 to 0.9, e.g., about 0.3, about 0.35, about 0.4, about 0.45, about 0.5, about 0.55, about 0.6, about 0.65, about 0.7, about 0.75, about 0.8, about 0.85, or about 0.9.

Additionally or alternatively, the liposomes can have a lipid composition characterized by a particular phase-transition temperature amenable for loading and delivery of ITE. In some embodiments, the average phase transition temperature of a lipid composition of a population of liposomes is from −60° C. to 80° C. (e.g., from −60° C. to −50° C., from −60° C. to −40° C., from −40° C. to −30° C., from −30° C. to −30° C., from −20° C. to −10° C., from −10° C. to 0° C., from 0° C. to 10° C., from 10° C. to 20° C., from 20° C. to 30° C., from 30° C. to 40° C., from 40° C. to 50° C., from 50° C. to 60° C., from 60° C. to 70° C., or from 70° C. to 80° C., e.g., about −60° C., about −50° C., about −40° C., about −30° C., about −20° C., about −10° C., about 0° C., about 10° C., about 20° C., about 30° C., about 40° C., about 50° C., about 60° C., about 70° C., or about 80° C.).

Tolerogenic liposomes can be made using any suitable method known in the art or described herein. In some embodiments, ITE is loaded into liposomes by micelle transfer (e.g., as described in Example 2). In other embodiments, ITE is loaded into liposomes by ethanol injection (e.g., as described in Example 3. For example, lipids can be combined in the desired ratio and dissolved in ethanol prior to addition of ITE in DMSO (e.g., from a stock concentration from 40-80 mg/mL, e.g., about 60 mg/mL). The volume of added ITE, in some embodiments, does not exceed 10% (e.g., does not exceed 9%, 8%, 7%, 6%, or 5%) of the volume of lipid/ethanol solution. ITE is able to dissolve into the lipid mixture in 10-30 minutes by heating at 35-40° C. with agitation. The resulting lipid/ITE/ethanol solution can then be transferred into a pre-warmed HBS solution containing antigen (e.g., at a concentration from 0.01 to 10 mg/mL, e.g., from 0.05 to 5 mg/mL, e.g., about 0.05 mg/mL, 0.10 mg/mL, 0.15 mg/mL, 0.20 mg/mL, 0.25 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, or 10 mg/mL). After stirring at 35-40° C. while allowing liposomes to form around the aqueous antigen solution, the mixture is passed through a series of polycarbonate membranes using conventional extrusion techniques to peel away multi-lamellar membranes and reduce the size (and size distribution) of liposomes. Passing the liposomes through 200 nm- and/or 100 nm-pore size membranes can yield nanoparticulate liposomes having a narrow distribution around 90-110 nm in diameter. Soluble antigen residing outside of the liposome core can be washed out of the liposome suspension by any suitable method (e.g., filtration, dialysis, or centrifugation).

### Antigens for Autoimmune Diseases

In some embodiments, the nanoparticles (e.g., nanoparticulate liposomes) provided herein include an antigen (e.g., within the aqueous core of the liposome). Nanoparticles may include any antigen associated with the pathogenic immune response against which the composition is configured to treat. For example, proteins which initiate autoimmune T and B cell reactions in rheumatoid arthritis patients include vimentin (including mutated vimentin), fibrinogen, alpha enolase, and collagen. Immune responses are frequently directed against post-translationally modified variants of these proteins. These most common protein modifications result from the enzymatic deimination of arginine (by peptidylarginine deiminases) to the non-standard amino acid citrulline. The use of citrullinated peptides for diagnosis of rheumatoid arthritis is well known. For example WO 2007/123976 discloses vimentin peptides, including citrullinated peptides, for diagnosis of rheumatoid arthritis, WO 2007/017556, discloses collagen type II peptides, including citrullinated peptides, for diagnosis of rheumatoid arthritis, and WO 2008/090360 discloses the use of enolase peptides, including citrullinated peptides, for diagnosis of rheumatoid arthritis. Rheumatoid arthritis-associated antigens that may be used as antigens of the present invention include proteins and peptides such as aggrecan, alpha enolase, collagen type II, fibrinogen, filaggrin, or vimentin. Relevant peptide sequences are provided in U.S. Patent Publication No. 2016/0024183. Each of the aforementioned publications are incorporated herein by reference in their entirety.

Other suitable antigens for various use in various indications include, for example, myelin basic protein, acetylcholine receptor, endogenous antigen, myelin oligodendrocyte glycoprotein (MOG), pancreatic beta-cell antigen, insulin, glutamic acid decarboxylase (GAD), collagen type II, human carticlage gp39, fp130-RAPS, proteolipid protein, fibrillarin, small nucleolar protein, thyroid stimulating factor receptor, histones, glycoprotein gp70, pyruvate dehydrogenase dehyrolipoamide acetyltransferase (PCD-E2), hair follicle antigen, or human tropomyosin isoform 5. Antigens can also be proteins containing polypeptides at least 80%, 85%, 90%, 95%, 96%, 97%, 98% or 99% identical to any of the above antigens, or fragments thereof.

### Antigens for Tolerizing to Peptide, Protein, and Gene Therapies

In some embodiments, the compositions and methods described herein feature nanoparticles (e.g., nanoparticulate liposomes) that include (e.g., encapsulate) an antigen (e.g., an antigen associated with a therapeutic agent, such as a therapeutic protein, viral vector or nanoparticle). Such embodiments can be used, e.g., to tolerize a subject to an antigen to which the subject may otherwise develop an adverse immune response.

In some embodiments, the antigen encapsulated in the nanoparticles (e.g., nanoparticulate liposomes) featured herein is a neoantigen. In some embodiments, the immunity to the neoantigen may pre-date therapeutic exposure. For example, adenoviruses, adeno-associated viruses (AAVs) and herpes viruses are ubiquitous. Thus when these viruses, or modified versions thereof, are used as gene therapy vectors, they may encounter pre-existing immunity, which can interfere with viral transduction. Also, re-exposure to the virus (in the form of gene therapy) often elicits a more robust immune response.

In some embodiments, the neoantigens can be a derived from any portion of a therapeutic agent (e.g., any portion of a peptide, protein, gene therapy or nanoparticle). When the specific structure (e.g., peptide sequence) of a neoantigen is not fully defined, a larger fragment of the therapeutic agent may be formulated in the nanoparticles (e.g., nanoparticulate liposome) to provide the neoantigen. Alternatively, the entire therapeutic peptide, protein, viral coat protein or virus may be encapsulated in the nanoparticle (e.g., nanoparticulate liposome).

In some embodiments, diseases or disorders that can be treated by a therapeutic agent described herein (e.g., a therapeutic agent comprising an antigen encapsulated in a nanoparticle (e.g., nanoparticulate liposome) of the present invention, which also carries ITE together with an inhibitor of ITE degradation (e.g., an esterase inhibitor)) may include, but are not limited to, autoimmune diseases, allergic diseases (e.g., allergies to environmental agents such as animal dander, pollen, dust mites, insect bites, and so forth, as well as food allergies to nuts, egg products, seafood, grains and so forth), hereditary diseases (treated by protein replacement therapy or gene therapy), organ transplantation (associated with immune rejection, or, in the case of bone marrow transplantation, with graft versus host disease), and a wide variety of other diseases treated by peptide or protein therapeutics (e.g., diabetes).

In some embodiments, autoimmune diseases that can be treated by a therapeutic agent (e.g., a therapeutic agent comprising an antigen encapsulated in a nanoparticle (e.g., nanoparticulate liposome) of the present invention, which also carries ITE together with an inhibitor of ITE degradation (e.g., an esterase inhibitor)), the compositions (e.g., nanoparticulate (e.g., nanoparticulate liposome) composition of the present invention) or methods described herein include, but are not limited to, diseases that affect the joints and connective tissues (e.g., ankylosing spondylitis, rheumatoid arthritis, juvenile idiopathic arthritis, psoriatic arthritis and mixed connective tissue disease), as well as diseases that affect the brain (e.g., multiple sclerosis and neuromyelitis optica), diseases that affect endocrine organs (e.g., type I diabetes, latent autoimmune diabetes in adults (LADA), autoimmune thyroid disease, Grave's disease, Hashimoto's thyroiditis and Addison's disease), diseases that affect the gastrointestinal tract (e.g., autoimmune atrophic gastritis, pernicious anemia, celiac disease, and inflammatory bowel disease (including Crohn's disease and ulcerative colitis)), diseases that affect the skin (e.g., psoriasis, scleroderma, alopecia areata, atopic dermatitis, pemphigus vulgaris and bullous pemphigoid), diseases that affect the muscle (e.g., myasthenia gravis, Guillain-Barre syndrome and poly/dermatomyositis), diseases that affect the heart (e.g., rheumatic fever), diseases that affect the liver (e.g., autoimmune hepatitis and primary sclerosing cholangitis), diseases that affect the sensory organs (e.g., autoimmune uveitis and Behcet's disease), diseases that affect the blood or bone marrow (e.g., autoimmune haemolytic anemia, idiopathic thrombocylopenic purpura and idiopathic leucopenia), diseases that affect the lungs (e.g., Goodpasture's syndrome), diseases that affect the kidney (e.g., autoimmune nephritis, including glomerulonephritis), diseases that affect the vasculature (e.g., Wegener's granulomatosis), diseases that affect the peripheral nervous system (e.g., chronic inflammatory demyelinating polyradiculoneuropathy), and diseases characterized by multi-organ involvement (e.g., systemic lupus erythematosus and Sjogren's syndrome).

In some embodiments, diseases or disorders that can be treated by a therapeutic agent (e.g., a therapeutic agent comprising an antigen encapsulated in a nanoparticle (e.g., nanoparticulate liposome) of the present invention, which also carries ITE together with an inhibitor of ITE degradation (e.g., an esterase inhibitor)), the compositions (e.g., nanoparticulate (e.g., nanoparticulate liposome) composition of the present invention) or methods described herein may include, but are not limited to, lysosomal storage diseases/ disorders, such as Santavuori-Haltia disease (Infantile Neuronal Ceroid Lipofuscinosis Type 1), Jansky-Bielschowsky Disease (late infantile neuronal ceroid lipofuscinosis, Type 2), Batten disease (juvenile neuronal ceroid lipofuscinosis, Type 3), Kufs disease (neuronal ceroid lipofuscinosis, Type 4), Von Gierke disease (glycogen storage disease, Type la), glycogen storage disease, Type Ib, Pompe disease (glycogen storage disease, Type II), Forbes or Cori disease (glycogen storage disease, Type III), mucolipidosis II (I-Cell disease), mucolipidosis III (Pseudo-Hurler polydystrophy), mucolipidosis IV (sialolipidosis), cystinosis (adult nonnephropathic type), cystinosis (infantile nephropathic type), cystinosis (juvenile or adolescent nephropathic), Salla disease/infantile sialic acid storage disorder, and saposin deficiencies; disorders of lipid and sphingolipid degradation, such as GM1 gangliosidosis (infantile, late infantile/juvenile, and adult/ chronic), Tay-Sachs disease, Sandhoff disease, GM2 gangliodisosis, Ab variant, Fabry disease, Gaucher disease, Types I, II and III, metachromatic leukidystrophy, Krabbe disease (early and late onset), Neimann-Pick disease, Types A, B, Cl, and C2, Farber disease, and Wolman disease (cholesteryl esther storage disease); disorders of mucopolysaccharide degradation, such as Hurler syndrome (MPSI), Scheie syndrome (MPS IS), Hurler-Scheie syndrome (MPS IH/S), Hunter syndrome (MPS II), Sanfillippo A syndrome (MPS IIIA), Sanfillippo B syndrome (MPS IIIB), Sanfillippo C syndrome (MPS IIIC), Sanfillippo D syndrome (MPS IIID), Morquio A syndrome (MPS IVA), Morquio B syndrome (MPS IVB), Maroteaux-Lamy syndrome (MPS VI), and Sly syndrome (MPS VII); disorders of glycoprotein degradation, such as alpha mannosidosis, beta mannosidosis, fucosidosis, asparylglucosaminuria, mucolipidosis I (sialidosis), galactosialidosis, Schindler disease, and Schindler disease, Type II/Kanzaki disease; and leukodystrophy diseases/disorders, such as abetalipoproteinemia, neonatal adrenoleukodystrophy, Canavan disease, cerebrotendinous xanthromatosis, Pelizaeus Merzbacher disease, Tangier disease, Refum disease (infantile and classic forms), acid maltase deficiency (e.g., Pompe disease, glycogenosis type 2, lysosomal storage disease), carnitine deficiency, camitine palmityl transferase deficiency, debrancher enzyme deficiency (e.g., Cori or Forbes disease, glycogenosis type 3), lactate dehydrogenase deficiency (e.g., glycogenosis type 11), myoadenylate deaminase deficiency, phosphofructokinase deficiency (e.g., Tarui disease, glycogenosis type 7), phosphogylcerate kinase deficiency (e.g., glycogenosis type 9), phosphogylcerate mutase deficiency (e.g., glycogenosis type 10), phosphorylase deficiency (e.g., McArdle disease, myophosphorylase deficiency, glycogenosis type 5), Gaucher's Disease (e.g., chromosome 1, enzyme glucocerebrosidase affected), Achondroplasia (e.g., chromosome 4, fibroblast growth factor receptor 3 affected), Huntington's Disease (e.g., chromosome 4, huntingtin), Hemochromatosis (e.g., chromosome 6, HFE protein), Cystic Fibrosis (e.g., chromosome 7, CFTR), Friedreich' s Ataxia (chromosome 9, frataxin), Best Disease (chromosome 11, VMD2), Sickle Cell Disease (chromosome 11, hemoglobin), Phenylketoniuria (chromosome 12, phenylalanine hydroxylase), Marfan's Syndrome (chromosome 15, fibrillin), Myotonic Dystophy (chromosome 19, dystophia myotonica protein kinase), Adrenoleukodystrophy (x-chromosome, lignoceroyl-CoA ligase in peroxisomes), Duchene's Muscular Dystrophy (x-chromosome, dystrophin), Rett Syndrome (x-chromosome, methylCpG-binding protein 2), Leber's Hereditary Optic Neuropathy (mitochondria, respiratory proteins), Mitochondria Encephalopathy, Lactic Acidosis and Stroke (MELAS) (mitochondria, transfer RNA), enzyme deficiencies of the Urea Cycle, Sickle Cell Anemia, Myotubular Myopathy, Hemophilia B, Lipoprotein lipase deficiency, Omithine Transcarbamylase Deficiency, Crigler-Najjar Syndrome, Mucolipidosis IV, Niemann-Pick A, San-filippo A, Sanfilippo B, Sanfilippo C, Sanfilippo D, beta-thalassaemia, Duchenne Muscular Dystrophy, and diseases or disorders that are the result of defects in lipid and sphingolipid degradation, muco-polysaccharide degradation, glycoprotein degradation, leukodystrophies, etc.

### Viral Vectors

Nanoparticle (e.g., nanoparticulate liposome) provided by the present invention can include (e.g., encapsulate) one or more viruses (e.g., viral vectors), or portion(s) thereof, such as capsid proteins. Examples of viral vectors that may be used (e.g., in therapeutic agents to which methods of the invention provide tolerance in a subject) are known in the art and/or described herein. Suitable viral vectors include, for instance, retroviral vectors, lentiviral vectors, herpes simplex virus (HSV)-based vectors, adenovirus-based vectors, adeno-associated virus (AAV)-based vectors, AAV-adenoviral chimeric vectors, vaccinia virus-based vectors and Sendai virus-based vectors.

In some embodiments, the viral vector described herein may be based on a retrovirus. Retroviruses (e.g., viruses belonging to the family Retroviridiae) have a single-stranded positive sense RNA genome and are capable of infecting a wide variety of host cells. Upon infection, the retroviral genome integrates into the genome of its host cell, using its own reverse transcriptase enzyme to produce DNA from its RNA genome. The viral DNA is then replicated along with host cell DNA, which translates and transcribes the viral and host genes. A retroviral vector can be manipulated to render the virus replication incompetent. As such, retroviral vectors are thought to be particularly useful for stable gene transfer in vivo. Examples of retroviral vectors useful in the present invention can be found, for example, in U.S. Publication Nos. 20120009161, 20090118212, and 20090017543, the viral vectors and methods of their making being incorporated by reference herein in their entirety.

Lentiviral vectors are examples of retroviral vectors that can be used for the production of a viral vector as provided herein. Lentiviruses have the ability to infect non dividing cells, a property that constitute a more efficient method of a gene delivery vector (see, e.g., Durand et al., Viruses. 2011 February; 3(2): 132-159). Examples of lentiviruses include HIV (humans), simian immunodeficiency virus (SIV), feline immunodeficiency virus (FIV), equine infectious anemia virus (EIAV) and visna virus (ovine lentivirus). Unlike other retroviruses, HIV-based vectors are known to incorporate their passenger genes into non-dividing cells. Examples of lentiviral vectors useful in the present invention can be found, for example, in U.S. Publication Nos. 20150224209, 20150203870, 20140335607, 20140248306, 20090148936, and 20080254008, the viral vectors and methods of their making being incorporated by reference herein in their entirety.

In some embodiments, Herpes simplex virus (HSV)-based viral vectors are suitable for use as provided herein. Many replication deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. For a description of HSV-based vectors useful in the present invention, see, for example, U.S. Pat. Nos. 5,837,532, 5,846,782, 5,849,572, and 5,804,413, and International Patent Applications WO 91/02788, WO 96/04394, WO 98/15637, and WO 99/06583, the description of which viral vectors and methods of their making being incorporated by reference in its entirety.

In some embodiments, the viral vector described herein may be based on Adenoviruses (Ads). Adenoviruses are non-enveloped viruses that can transfer DNA in vivo to a variety of different target cell types. The virus can be made replication-deficient by deleting select genes required for viral replication. The expendable non-replication-essential E3 region is also frequently deleted to allow additional room for a larger DNA insert. Adenoviral vectors can be produced in high titers and can efficiently transfer DNA to replicating and non-replicating cells. Unlike lentivirus, adenoviral DNA does not integrate into the genome and therefore is not replicated during cell division, instead they replicate in the nucleus of the host cell using the host's replication machinery.

The adenovirus on which a viral vector featured herein may be based, may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, VA). Non-group C adenoviruses, and even non-human adenoviruses, can be used to prepare replication deficient adenoviral vectors. Non-group C adenoviral vectors, methods of producing non-group C adenoviral vectors, and methods of using non-group C adenoviral vectors useful in the present invention are disclosed in, for example, U.S. Pat. Nos. 5,801,030, 5,837,511, and 5,849,561, and International Patent Applications WO 97/12986 and WO 98/53087. Any adenovirus, even a chimeric adenovirus, can be used as the source of the viral genome for an adenoviral vector. For example, a human adenovirus can be used as the source of the viral genome for a replication deficient adenoviral vector. Further examples of adenoviral vectors useful in the present invention can be found in U.S. Publication Nos. 20150093831, 20140248305, 20120283318, 20100008889, 20090175897 and 20090088398, the description of which viral vectors and methods of their making being incorporated by reference in its entirety.

In some embodiments, the viral vectors provided herein can also be based on AAVs. AAV vectors have been of particular interest for use in therapeutic applications such as those described herein. AAV is a DNA virus, which is not known to cause human disease. Generally, AAV requires co-infection with a helper virus (e.g., an adenovirus or a herpes virus), or expression of helper genes, for efficient replication. AAVs have the ability to stably infect host cell genomes at specific sites, making them more predictable than retroviruses; however, generally, the cloning capacity of the vector is 4.9 kb. AAV vectors that have been used in gene therapy applications generally have had approximately 96% of the parental genome deleted, such that only the terminal repeats (ITRs), which contain recognition signals for DNA replication and packaging, remain. For a description of AAV-based vectors useful in the present invention, see, for example, U.S. Pat. Nos. 8,679,837, 8,637,255, 8,409,842, 7,803,622, and 7,790,449, and U.S. Publication Nos. 0150065562, 20140155469, 20140037585, 20130096182, 20120100606, and 20070036757, the viral vectors of which and methods or their making being incorporated herein by reference in their entirety. The AAV vectors may be recombinant AAV vectors. The AAV vectors may also be self-complementary (sc) AAV vectors, which are described, for example, in U.S. Patent Publications 2007/01110724 and 2004/0029106, and U.S. Pat. Nos. 7,465,583 and 7,186,699, the vectors and methods of production of which are herein incorporated by reference.

In some embodiments, the viral vector (e.g., AAV vector) included in the methods and compositions described herein is configured to deliver a RNA molecule. In some embodiments, the viral vector (e.g., AAV vector) included in the methods and compositions described herein is configured to deliver a small interfering RNA (siRNA). In some embodiments, the viral vector (e.g., AAV vector) included in the methods and compositions described herein is configured to deliver a microRNA (miRNA). In some embodiments, the viral vector (e.g., AAV vector) included in the methods and compositions described herein is configured to deliver a short hairpin RNA (shRNA). In some embodiments, the viral vector (e.g., AAV vector) included in the methods and compositions described herein is configured to deliver an mRNA. In some embodiments, the viral vector (e.g., AAV vector) included in the methods and compositions described herein is configured to deliver a small activating RNA (saRNA).

In some embodiments, the viral vector (e.g., AAV vector) included in the methods and compositions described herein is configured to deliver a DNA molecule. In some embodiments, the viral vector (e.g., AAV vector) included in the methods and compositions described herein is configured to deliver a single stranded DNA (ssDNA). In some embodiments, the ssDNA encodes a cDNA or a fragment thereof. In some embodiments, the cDNA is a human cDNA or a fragment thereof.

The AAV on which a viral vector may be based can be of any serotype or a mixture of serotypes. AAV serotypes include AAVI, AAV 2, AAV3, AAV4, AAVS, AAV6, AAV7, AAV8, AAV9, AAVIO, and AAVII. For example, when the viral vector is based on a mixture of serotypes, the viral vector may contain the capsid signal sequences taken from one AAV serotype (for example, selected from any one of AAV serotypes 1, 2, 3, 4, 5, 6, 7, 8, 9, 10, and 11) and packaging sequences from a different serotype (for example, selected from any one of AAV serotypes 1, 2, 3, 4, 5, 6, 7, 8, 9, 10, and 11). In some embodiments of any one of the methods or compositions provided herein, therefore, the AAV vector is an AAV 2/8 vector. In other embodiments of any one of the methods or compositions provided herein, the AAV vector is an AAV 2/5 vector. As an alternative to formulating AAV particles, specific AAV viral proteins or peptides known to contain immunogenic epitopes can be used to induce tolerance. For example, recombinant AAV capsid proteins VP1, VP2 or VP3 (associated with any AAV serotype, native or engineered) can be formulated in liposomes with ITE. Alternatively, peptides encompassing T cell epitopes known to elicit immunity (e.g. as identified by Manno et al. Nature Medicine 2006, 12(3): 342-347) can be formulated in liposomes with ITE, for example, peptides 74 (PADVFMVPQYGYLTL) and 82 (GNNFTFSYTFEDVPF) and AAV serotype-specific variants thereof.

In some embodiments, the viral vectors provided herein may be based on an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Masso das Pedras virus, Mucambo virus, Ndumu virus, O′nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819; the vectors and methods of their making are incorporated herein by reference in their entirety. Viral vectors can be used to deliver transgenes for a variety of purposes, including for gene editing, the methods and compositions provided herein are also so applicable.

### Viral Vector: Therapeutic Protein or Portion Thereof

In some embodiments, a viral vector may include a transgene that encodes a therapeutic protein or portion thereof as provided herein. Examples of such proteins include, but are not limited to, infusible or injectable therapeutic proteins, enzymes, enzyme cofactors, hormones, blood or blood coagulation factors, cytokines and interferons, growth factors, adipokines, monoclonal antibodies, etc.

In some embodiments, a viral vector may include a transgene that encodes an infusible or injectable therapeutic protein. Examples of such infusible or injectable therapeutic proteins include, but are not limited to, Tocilizumab (Roche/Actemra®), alpha-1 antitrypsin (Kamada/AAT), Hematide® (Affymax and Takeda, synthetic peptide), albinterferon alfa-2b (Novar-tis/Zalbin™), Rhucin® (Pharming Group, CI inhibitor replacement therapy), tesamorelin (Theratechnologies/ Egrifta, synthetic growth hormone-releasing factor), ocrelizumab (Genentech, Roche and Biogen), belimumab (Glaxo-SmithKline/Benlysta®), pegloticase (Savient Pharmaceuticals/Krystexxa™), taliglucerase alfa (Protalix/ Uplyso), agalsidase alfa (Shire/Replagal®), and velaglucerase alfa (Shire).

In some embodiments, a viral vector may include a transgene that encodes an enzyme (e.g., for an enzyme replacement therapy). Examples of such enzymes include, but are not limited to, lysozyme, oxidoreductases, transferases, hydrolases, lyases, isomerases, asparaginases, uricases, glycosidases, proteases, nucleases, collagenases, hyaluronidases, heparinases, heparanases, kinases, phosphatases, lysins and ligases. Other examples of enzymes include those that used for enzyme replacement therapy including, but not limited to, imiglucerase (e.g., CEREZYME™), a-galactosidase A (a-gal A) (e.g., agalsidase beta, FABRYZYME), acid a-glucosidase (GAA) (e.g., alglucosidase alfa, LUMIZYME™, MYOZYME™), and arylsulfatase B (e.g., laronidase, ALDURAZYME™, idursulfase, ELAPRASE™ arylsulfatase B, NAGLAZYME™).

In some embodiments, a viral vector may include a transgene that encodes a hormone. Examples of such hormones include, but are not limited to, Melatonin (N-acety1-5-methoxytryptamine), Serotonin, Thyroxine (or tetraiodothyronine) (a thyroid hormone), Triiodothyronine (a thyroid hormone), Epinephrine (or adrenaline), Norepinephrine (or noradrenaline), Dopamine (or prolactin inhibiting hormone), Antimullerian hormone (or mullerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Calcitonin, Cholecystokinin, Corticotropin-releasing hormone, Erythropoietin, Follicle-stimulating hormone, Gastrin, Ghrelin, Glucagon, Glucagon-like peptide (GLP-1), GIP, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Luteinizing hormone, Melanocyte stimulating hormone, Orexin, Oxytocin, Parathyroid hormone, Prolactin, Relaxin, Secretin, Somatostatin, Thrombopoietin, Thyroid-stimulating hormone (or thyrotropin), Thyrotropin-releasing hormone, Cortisol, Aldosterone, Testosterone, Dehydroepiandrosterone, Androstenedione, Dihydrotestosterone, Estradiol, Estrone, Estriol, Progesterone, Calcitriol (1,25-dihydroxyvitamin D3), Calcidiol (25-hydroxyvitamin D3), Prostaglandins, Leukotrienes, Prostacyclin, Thromboxane, Prolactin releasing hormone, Lipotropin, Brain natriuretic peptide, Neuropeptide Y, Histamine, Endothelin, Pancreatic polypeptide, Renin, and Enkephalin.

In some embodiments, a viral vector may include a transgene that encodes a blood or blood coagulation factor. Examples of such blood or blood coagulation factors include, but are not limited to, Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, von Heldebrant Factor, prekallikrein (Fletcher factor), high-molecular weight kininogen (HMWK) (Fitzgerald factor), fibronectin, fibrin, thrombin, antithrombin, such as antithrombin III, heparin cofactor II, protein C, protein S, protein Z, protein Z-related protease inhibitor (ZPI), plasminogen, alpha 2-antiplasmin, tissue plasminogen activator (tPA), urokinase, plasminogen activator inhibitor-I (PAli), plasminogen activator inhibitor-2 (PAI2), cancer procoagulant, and epoetin alfa (Epogen, Procrit).

In some embodiments, a viral vector may include a transgene that encodes a cytokine. Examples of such cytokines include, but are not limited to, lymphokines, interleukins, and chemokines, type 1 cytokines, such as IFN-y, TGF-b, and type 2 cytokines, such as IL-4, IL-10, and IL-13.

In some embodiments, a viral vector may include a transgene that encodes a growth factor. Examples of such growth factors include, but are not limited to, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-b), Tumor necrosis factor-alpha (TNF-α), Vascular endothelial growth factor (VEGF), Wnt Signaling Pathway, placental growth factor (PIGF), [(Foetal Bovine Somatotrophin)] (FBS), IL-1, IL-2, IL-3, IL-4, IL-5, IL-6, and IL-7.

In some embodiments, a viral vector may include a transgene that encodes an adipokine. Examples of such adipokines, include, but are not limited to, leptin and adiponectin.

In some embodiments, a viral vector may include a transgene that encodes one or more of, receptors, signaling proteins, cytoskeletal proteins, scaffold proteins, transcription factors, structural proteins, membrane proteins, cytosolic proteins, binding proteins, nuclear proteins, secreted proteins, golgi proteins, endoplasmic reticulum proteins, mitochondrial proteins, and vesicular proteins, etc.

Additionally, a viral vector may include a transgene that encodes other therapeutic proteins, such as functional versions of proteins associated with disorders of lipid and sphingolipid degradation (e.g., Galactosidase-1, Hexosaminidase A, Hexosaminidases A and B, GM2 Activator Protein, 8-Galactosidase A, Glucocerebrosidase, Glucocerebrosidase, Glucocerebrosidase, Arylsulfatase A, Galactosylceramidase, Sphingomyelinase, Sphingomyelinase, NPCI, Acid Ceramidase, Lysosomal Acid Lipase); disorders of mucopolysaccharide degradation (e.g., L-Iduronidase, Iduronate Sulfatase, Heparan N-Sulfatase, N-Acetylglucosaminidase, Acetyl-CoA-Glucosaminidase, Acetyltransferase, Acetylglucosamine-6-Sulfatase, Galactosamine-6-Sulfatase, Arylsul-fatase B, Glucuronidase); disorders of glycoprotein degradation (e.g., Mannosidase, mannosidase, 1-fucosidase, Aspartylglycosaminidase, Neuraminidase, Lysosomal protective protein, Lysosomal 8-N-acetylgalactosaminidase, Lysosomal 8-N-acetylgalactosaminidase); lysosomal storage disorders (e.g., Palmitoyl-protein thioesterase, at least 4 subtypes, Lysosomal membrane protein, Unknown, Glucose-6-phosphatase, Glucose-6-phosphate translocase, Acid maltase, Debrancher enzyme amylo-1,6 glucosidase, N-acetylglucosamine-1-phosphotransferase, N-acetylglucosamine-1-phosphotransferase, Ganglioside sialidase (neuraminidase), Lysosomal cystine transport protein, Lysosomal cystine transport protein, Lysosomal cystine transport protein, Sialic acid transport protein Saposins, A, B, C, D) and leukodystrophies (e.g., Microsomal triglyceride transfer protein/apolipoprotein B, Peroxisomal membrane transfer protein, Peroxins, Aspartoacylase, Sterol-27-hydroxlase, Proteolipid protein, ABC transporter, Peroxisome membrane protein 3 or Peroxisome biogenesis factor 1, Phytanic acid oxidase).

### Viral Vector: Gene Editing

In some embodiments, the viral vectors described herein (e.g., viral vectors that may be used as therapeutic agents to which tolerance can be induced using the methods and compositions of the present invention) may be used for gene editing. In such embodiments, the transgene of the viral vector is a gene editing transgene. Such a transgene encodes a component that is involved in a gene editing process. Generally, such a process results in long-lasting or permanent modifications to genomic DNA, such as targeted DNA insertion, replacement, mutagenesis or removal. Gene editing may include the delivery of nucleic acids encoding a DNA sequence of interest and inserting the sequence of interest at a targeted site in genomic DNA using endonucleases. Thus, gene editing transgenes may comprise these nucleic acids encoding a DNA sequence of interest for insertion. In some embodiments, the DNA sequence for insertion is a DNA sequence encoding any one of the therapeutic proteins provided herein or a portion thereof. Alternatively, or in addition to, the gene editing transgene may comprise nucleic acids that encode one of more components that carry out the gene editing process. The gene editing transgenes provided herein may encode an endonuclease and/or a guide RNA, etc.

Endonucleases can create breaks in double-stranded DNA at desired locations in a genome and use the host cell's mechanisms to repair the break using homologous recombination, nonhomologous end-joining, etc. Classes of endonucleases that can be used for gene editing include, but are not limited to, meganucleases, zinc-finger nucleases (ZFNs), transcription activator-like effector nucleases (TALENs), clustered regularly interspaced short palindromic repeat(s) (CRISPR) and homing endonucleases. The gene editing transgene of the viral vectors provided herein may encode any one of the endonucleases provided herein.

In some embodiments, a viral vector may include a transgene that encodes a meganuclease. Meganucleases are generally characterized by their capacity to recognize and cut DNA sequences (14-40 base pairs). In addition, known techniques, such as mutagenesis and high-throughput screening and combinatorial assembly, can be used to create custom meganucleases, where protein subunits can be associated or fused. Examples of meganucleases can be found in U.S. Pat. Nos. 8,802,437, 8,445,251 and 8,338,157; and U.S. Publication Nos. 20130224863, 20110113509 and 20110033935, the meganucleases of which are incorporated herein by reference.

In some embodiments, a viral vector may include a transgene that encodes a zinc finger nuclease. A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Zinc finger nucleases can be designed to target virtually any desired sequence in a given nucleic acid molecule for cleavage. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo CO (May 1991). “Zinc finger-DNA recognition: crystal structure of a Zif268-DNA complex at 2.1 A”. Science 252 (5007): 809-17, the entire contents of which are incorporated herein). In some embodiments, bacterial or phage display is employed to develop a zinc finger domain that recognizes a desired nucleic acid sequence, for example, a desired endonuclease target site. Zinc finger nucleases, in some embodiments, comprise a zinc finger binding domain and a cleavage domain fused or otherwise conjugated to each other via a linker, for example, a polypeptide linker. The length of the linker can determine the distance of the cut from the nucleic acid sequence bound by the zinc finger domain. Examples of zinc finger nucleases can be found in U.S. Pat. Nos. 8,956,828; 8,921,112; 8,846,578; 8,569,253, the zinc finger nucleases of which are incorporated herein by reference.

In some embodiments, a viral vector may include a transgene that encodes a transcription activator-like effector nucleases (TALEN). TALENs are artificial restriction enzymes produced by fusing specific DNA binding domains to generic DNA cleaving domains. The DNA binding domains, which can be designed to bind any desired DNA sequence, come from transcription activator-like (TAL) effectors, DNA-binding proteins excreted by certain bacteria that infect plants. Transcription activator-like effectors (TALEs) can be engineered to bind practically any DNA sequence or joined together into arrays in combination with a DNA cleavage domain. TALENs can be used similarly to design zinc finger nucleases. Examples of TALENS can be found in U.S. Pat. No. 8,697,853; as well as U.S. Publication Nos. 20150118216, 20150079064, and 20140087426, the TALENS of which are incorporated herein by reference.

The CRISPR (clustered regularly interspaced short palindromic repeats)/Cas system can also be used as a tool for gene editing. In a CRISPR/Cas system, guide RNA (gRNA) is encoded genomically or episomally (e.g., on a plasmid). The gRNA forms a complex with an endonuclease, such as Cas9 endonuclease, following transcription. The complex is then guided by the specificity determining sequence (SDS) of the gRNA to a DNA target sequence, typically located in the genome of a cell. Cas9 or Cas9 endonuclease refers to an RNA-guided endonuclease comprising a Cas9 protein, or a fragment thereof (e.g., a protein comprising an active or inactive DNA cleavage domain of Cas9 or a partially inactive DNA cleavage domain (e.g., a Cas9 nickase), and/or the gRNA binding domain of Cas9). Cas9 recognizes a short motif in the CRISPR repeat sequences (the PAM or protospacer adjacent motif) to help distinguish self versus non-self. Cas9 endonuclease sequences and structures are well known to those of skill in the art (see, e.g., “Complete genome sequence of an MI strain of Streptococcus pyo-genes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L. expand/collapse author list McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guidedDNAendo-nuclease in adaptive bacterial immunity.” Jinek M., Chylinski K., Fonfara I., Hauer M., Doudna J. A., Charpentier E. Science 337:816-821(2012)). Single guide RNAs (“sgRNA”, or simply “gNRA”) can be engineered so as to incorporate aspects of both the crRNA and tracrRNA into a single RNA species. See e.g., Jinek M., Chylinski K., Fonfara I., Hauer M., Doudna J. A., Charpentier E. Science 337:816-821 (2012). In some embodiments, a viral vector may include a transgene that encodes a Cas9 endonuclease.

Cas9 orthologs have been described in various species, including, but not limited to, S. pyogenes and S. thermophilus. Additional suitable Cas9 endonucleases and sequences will be apparent to those of skill in the art, and such Cas9 endonucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737. In some embodiments, a gene editing transgene encodes a wild-type Cas9, fragment or a Cas9 variant. A “Cas9 variant” is any protein with a Cas9 function that is not identical to a Cas9 wild-type endonuclease as it occurs in nature. In some embodiments, a Cas9 variant shares homology to a wild-type Cas9, or a fragment thereof. A Cas9 variant in some embodiments has at least 40% sequence identity to Streptococcus pyogenes or S. thermophilus Cas9 protein and retains the Cas9 functionality. Preferably, the sequence identity is at least 90%, 95%, or more. More preferably, the sequence identity is at least 98% or 99% sequence identity. In some embodiments of any one of the Cas9 variants for use in any one of the methods provided herein the sequence identity is amino acid sequence identity. Cas9 variants also include Cas9 dimers, Cas9 fusion proteins, Cas9 fragments, minimized Cas9 proteins, Cas9 variants without a cleavage domain, Cas9 variants without a gRNA domain, Cas9-recombinase fusions, fCas9, Fokl-dCas9, etc. Examples of such Cas9 variants can be found, for example, in U.S. Publication Nos. 20150071898 and 20150071899, the description of Cas9 proteins and Cas9 variants of which is incorporated herein by reference. Cas9 variants also include Cas9 nickases, which comprise mutation(s) which inactivate a single endonuclease domain in Cas9. Such nickases can induce a single strand break in a target nucleic acid as opposed to a double strand break. Cas9 variants also include Cas9 null nucleases, a Cas9 variant in which one nuclease domain is inactivated by a mutation. Examples of additional Cas9 variants and/or methods of identifying further Cas9 variants can be found in U.S. Publication Nos. 20140357523, 20150165054 and 20150166980, the contents of which pertaining to Cas9 proteins, Cas9 variants and methods of their identification being incorporated herein by reference.

Still other examples of Cas9 variants include a mutant form, known as Cas9D10A, with only nickase activity. Cas9D10A is appealing in terms of target specificity when loci are targeted by paired Cas9 complexes designed to generate adjacent DNA nicks. Another example of a Cas9 variant is a nuclease-deficient Cas9 (dCas9). Mutations H840A in the HNH domain and D10A in the RuvC domain inactivate cleavage activity, but do not prevent DNA binding. Therefore, this variant can be used to sequence-specifically target any region of the genome without cleavage. Instead, by fusing with various effector domains, dCas9 can be used either as a gene silencing or activation tool. The gene editing transgene, in some embodiments, may encode any one of the Cas9 variants provided herein.

Methods of using RNA-programmable endonucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W.Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J.E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013)).

In some embodiments, a viral vector may include a transgene that encodes a homing endonuclease. Homing endonucleases can catalyze, at few or singular locations, the hydrolysis of the genomic DNA used to synthesize them, thereby transmitting their genes horizontally within a host, increasing their allele frequency. Homing endonucleases generally have long recognition sequences, they thereby have low probability of random cleavage. One allele carries the gene (homing endonuclease gene+, HEG+), prior to transmission, while the other does not (HEG-), and is susceptible to enzyme cleavage. The enzyme, once synthesized, breaks the chromosome in the HEG-allele, initiating a response from the cellular DNA repair system which takes the pattern of the opposite, using recombination, undamaged DNA allele, HEG+that contains the gene for the endonuclease. Thus, the gene is copied to another allele that initially did not have it, and it is propagated through successively. Examples of homing endonucleases can be found, for example, in U.S. Publication No. 20150166969; and U.S. Pat. No. 9,005,973, the homing endonucleases of which are incorporated herein by reference.

The sequence of a transgene may also include an expression control sequence. Expression control DNA sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include promoter sequences, e.g., cytomegalovirus promoter; Rous sarcoma virus promoter; and simian virus 40 promoter; as well as any other types of promoters that are disclosed elsewhere herein or are otherwise known in the art. Generally, promoters are operatively linked upstream (i.e., 5′) of the sequence coding for a desired expression product. The transgene also may include a suitable polyadenylation sequence (e.g., the SV40 or human growth hormone gene polyadenylation sequence) operably linked downstream (i.e., 3′) of the coding sequence.

### Antigen to ITE Stoichiometry

Various antigen-to-ITE ratios can be used, depending on the antigen and type of disease being treated. In some instances, the mass ratio of antigen-to-ITE is from 1:100 to 100:1 (e.g., from 1:50 to 50:1, from 1:20 to 20:1, or from 1:10 to 10:1, e.g., from 1:100 to 1:80, from 1:80 to 1:60, from 1:60 to 1:50, from 1:50 to 1:40, from 1:40 to 1:30, from 1:30 to 1:20, from 1:20 to 1:10, from 1:10 to 1:5, from 1:5 to 1:4, from 1:4 to 1:3, from 1:3 to 1:2, from 1:2 to 1:1, from 1:1 to 2:1, from 2:1 to 3:1, from 3:1 to 4:1, from 4:1 to 5:1, from 5:1 to 10:1, from 10:1 to 20:1, from 20:1 to 30:1, from 30:1 to 40:1, from 40:1 to 50:1, from 50:1 to 60:1, from 60:1 to 80:1, or from 80:1 to 100:1, e.g., about 1:100, 1:90, 1:80, 1:70, 1:60, 1:50, 1:40, 1:30, 1:20:1:10, 1:5, 1:4,1:3,1:2,1:1,2:1,3:1,4:1,5:1, 10:1, 20:1, 30:1, 40:1, 50:1, 60:1, 70:1, 80:1, 90:1, or 100:1). In some embodiments, the molar ratio of antigen-to-ITE is from 1:100 to 100:1 (e.g., from 1:50 to 50:1, from 1:20 to 20:1, or from 1:10 to 10:1, e.g., from 1:100 to 1:80, from 1:80 to 1:60, from 1:60 to 1:50, from 1:50 to 1:40, from 1:40 to 1:30, from 1:30 to 1:20, from 1:20 to 1:10, from 1:10 to 1:5, from 1:5 to 1:4, from 1:4 to 1:3, from 1:3 to 1:2, from 1:2 to 1:1, from 1:1 to 2:1, from 2:1 to 3:1, from 3:1 to 4:1, from 4:1 to 5:1, from 5:1 to 10:1, from 10:1 to 20:1, from 20:1 to 30:1, from 30:1 to 40:1, from 40:1 to 50:1, from 50:1 to 60:1, from 60:1 to 80:1, or from 80:1 to 100:1, e.g., about 1:100, 1:90, 1:80, 1:70, 1:60, 1:50, 1:40, 1:30, 1:20: 1:10, 1:5, 1:4, 1:3, 1:2, 1:1, 2:1, 3:1, 4:1, 5:1, 10:1, 20:1, 30:1, 40:1, 50:1, 60:1, 70:1, 80:1, 90:1, or 100:1).

### Methods of Treatment and Prevention

The compositions and methods described herein are of particular use for treating a patient (e.g., a human) that would benefit from therapeutic immunomodulation (e.g., a patient in need of a suppressed immune response, e.g., a subject having an autoimmune disorder) or for preventing the onset of development of a disorder (e.g., an autoimmune disorder). The autoimmune disorder to be treated or prevented can be an autoimmune skin disease, such as alopecia areata, bullous pemphigoid, dermatomyositis, epidermolysis bullosa, pemphigus vulgaris, psoriasis or scleroderma, or vitiligo. The methods include selecting a patient in need of treatment and administering to the patient one or more of the compositions described herein. A subject in need of treatment can be identified, e.g., by their medical practitioner.

In some embodiments, diseases or disorders that can be treated by the compositions (e.g., nanoparticulate (e.g., nanoparticulate liposome) composition of the present invention) and methods described herein may include, but are not limited to, autoimmune diseases, allergic diseases (e.g., allergies to environmental agents such as animal dander, pollen, dust mites, insect bites, and so forth, as well as food allergies to nuts, egg products, seafood, grains and so forth), hereditary diseases (treated by protein replacement therapy or gene therapy), organ transplantation (associated with immune rejection, or, in the case of bone marrow transplantation, with graft versus host disease), and a wide variety of other diseases treated by peptide or protein therapeutics (e.g., diabetes).

In some embodiments, autoimmune diseases that can be treated by the compositions (e.g., nanoparticulate (e.g., nanoparticulate liposome) composition of the present invention) or methods described herein include, but are not limited to, diseases that affect the joints and connective tissues (e.g., ankylosing spondylitis, rheumatoid arthritis, juvenile idiopathic arthritis, psoriatic arthritis and mixed connective tissue disease), as well as diseases that affect the brain (e.g., multiple sclerosis and neuromyelitis optica), diseases that affect endocrine organs (e.g., type I diabetes, latent autoimmune diabetes in adults (LADA), autoimmune thyroid disease, Grave's disease, Hashimoto's thyroiditis and Addison's disease), diseases that affect the gastrointestinal tract (e.g., autoimmune atrophic gastritis, pernicious anemia, celiac disease, and inflammatory bowel disease (including Crohn's disease and ulcerative colitis)), diseases that affect the skin (e.g., psoriasis, scleroderma, alopecia areata, atopic dermatitis, pemphigus vulgaris and bullous pemphigoid), diseases that affect the muscle (e.g., myasthenia gravis, Guillain-Barre syndrome and poly/dermatomyositis), diseases that affect the heart (e.g., rheumatic fever), diseases that affect the liver (e.g., autoimmune hepatitis and primary sclerosing cholangitis), diseases that affect the sensory organs (e.g., autoimmune uveitis and Behcet's disease), diseases that affect the blood or bone marrow (e.g., autoimmune haemolytic anemia, idiopathic thrombocylopenic purpura and idiopathic leucopenia), diseases that affect the lungs (e.g., Goodpasture's syndrome), diseases that affect the kidney (e.g., autoimmune nephritis, including glomerulonephritis), diseases that affect the vasculature (e.g., Wegener's granulomatosis), diseases that affect the peripheral nervous system (e.g., chronic inflammatory demyelinating polyradiculoneuropathy), and diseases characterized by multi-organ involvement (e.g., systemic lupus erythematosus and Sjogren's syndrome).A therapeutically effective amount of one or more of the compositions described herein can be administered by standard methods, for example, by one or more routes of administration, e.g., by one or more of the routes of administration currently approved by the United States Food and Drug Administration (FDA; see, for example world wide web address fda.gov/cder/dsm/DRG/drg00301.htm). Tolerogenic nanoparticles or pharmaceutical compositions thereof can be administered systemically or locally, e.g., parenterally via intravenous, subcutaneous, intradermal (including intra-epidermal), dermal, intra-articular, pulmonary or mucosal (including nasal) routes. In some embodiments, compositions may be administered intrahepatically, intracerebrally, intramuscularly, percutaneously, intraarterially, intraperitoneally, intralesionally, intracranially, intraarticularly, intraprostatically, intrapleurally, intratracheally, intrathecally, intranasally, intravaginally, intrarectally, intratumorally, subcutaneously, subconjunctivally, intravesicularly, mucosally, intrapericardially, intraumbilically, orally, topically, transdermally, by inhalation, by aerosolization, by injection, by implantation, by infusion (e.g., by continuous infusion), by catheter, by lavage, or in creams.

The amount of liposomes administered to the patient may vary depending on the concentration of antigen, ITE, and/or inhibitor of ITE. In some embodiments, the amount of ITE in a single dose of nanoparticles is between about 1 μg and 10 mg (e.g., from 10 μg to 10 mg, from 50 μg to 5 mg, from 100 μg to 1 mg, from 150 μg to 500 μg, from 200 μg to 400 μg, or from 250 μg to 250 μg, e.g., from 1 μg to 10 μg, from 10 μg to 50 μg, from 50 μg to 100 μg, from 100 μg to 150 μg, from 150 μg to 200 μg, from 200 μg to 250 μg, from 250 μg to 300 μg, from 300 μg to 350 μg, form 350 μg to 400 μg, from 400 μg to 450 μg, from 450 μg to 500 μg, from 500 μg to 600 μg, from 600 μg to 700 μg, from 700 μg to 800 μg, from 800 μg to 900 μg, from 900 μg to 1.0 mg, from 1.0 mg to 5.0 mg, or from 5.0 mg to 10 mg). For example, the amount of ITE in a single dose of liposomes can be from 100 μg to 500 μg (e.g., from 200 μg to 300 μg, e.g., about 200 μg, about 210 μg, about 220 μg, about 230 μg, about 240 μg, about 250 μg, about 260 μg, about 270 μg, about 280 μg, about 290 μg, or about 300 μg).

In some embodiments, the amount of inhibitor of ITE in a single dose of nanoparticles is between about 1 μg and 50 mg (e.g., from 10 μg to 50 mg, from 50 μg to 20 mg, from 100 μg to 10 mg, from 150 μg to 1 mg, from 200 μg to 500 μg, or from 250 μg to 250 μg, e.g., from 1 μg to 10 μg, from 10 μg to 50 μg, from 50 μg to 100 μg, from 100 μg to 150 μg, from 150 μg to 200 μg, from 200 μg to 250 μg, from 250 μg to 300 μg, from 300 μg to 350 μg, form 350 μg to 400 μg, from 400 μg to 450 μg, from 450 μg to 500 μg, from 500 μg to 600 μg, from 600 μg to 700 μg, from 700 μg to 800 μg, from 800 μg to 900 μg, from 900 μg to 1.0 mg, from 1.0 mg to 5.0 mg, from 5.0 mg to 10 mg, from 10 mg to 20 mg, from 20 mg to 30 mg, from 30 mg to 40 mg, or from 40 mg to 50 mg). For example, the amount of ITE in a single dose of liposomes can be from 100 μg to 500 μg (e.g., from 200 μg to 300 μg, e.g., about 200 μg, about 210 μg, about 220 μg, about 230 μg, about 240 μg, about 250 μg, about 260 μg, about 270 μg, about 280 μg, about 290 μg, or about 300 μg). In some embodiments, the inhibitor of ITE is rivastigmine, and the amount of rivastigmine in a single dose of nanoparticles is from 1 mg to 12 mg (e.g., from 1 mg to 12 mg, from 2 mg to 10 mg, from 3 mg to 8 mg, or from 4 mg to 7 mg). Pharmaceutical compositions of tolerogenic nanoparticles can be of any suitable concentration for the intended route of administration.

As an alternative to administration of nanoparticles co-formulated with ITE and an inhibitor thereof, a population of nanoparticles containing ITE (e.g., with or without an antigen) can be administered separately from the inhibitor of ITE degradation. For example, the population of nanoparticles containing ITE can be administered at separate times, at separate frequencies, and/or by separate routes of administration than the ITE degradation inhibitor. In some embodiments, the ITE degradation inhibitor is administered systemically. In some embodiments, the ITE degradation inhibitor is administered locally, e.g., topically (e.g., by cream or lotion) or transdermally (e.g., by patch).

In some embodiments, a single unit dose of tolerogenic nanoparticles (e.g., a single dose containing any of the aforementioned amounts of ITE and/or inhibitor of ITE degradation) can include from 10¹¹ to 10¹⁶ nanoparticles (e.g., from 10¹¹ to 10¹⁶ nanoparticles, from 10¹¹ to 10¹⁵ nanoparticles, from 10¹² to 10¹⁴ nanoparticles, or about 10¹³ nanoparticles, e.g., from 10¹¹ to 10¹² nanoparticles, from 10¹² to 10¹³ nanoparticles, from 10¹³ to 10¹⁴ nanoparticles, from 10¹⁴ to 10¹⁵ nanoparticles, or from 10¹⁵ to 10¹⁶ nanoparticles, e.g., from 10¹² to 10¹⁵ nanoparticles, or from 5.0×10¹³ to 1.0×10¹⁴ nanoparticles, e.g., about 8×10¹³ nanoparticles).

The effective dose of the inhibitor may be selected according to the formulation and method of administration, and can further be calibrated to provide a desired level of in vivo inhibition of ITE degradation. In some embodiments, ITE degradation, as measured by its in vitro or in vivo half life, is extended by at least two-fold (e.g. from about 10 minutes to about 20 minutes), e.g., at least three-fold, at least four-fold, at least five-fold, at least ten-fold, at least twenty-fold, at least forty-fold, or at least fifty-fold, or any intermediate value. Depending on the formulation and route of administration of the inhibitor of ITE degradation, ITE half life extension may only be observable in certain cell or tissue compartments, such as those cell or tissue types which concentrate nanoparticles, or in the case of dermal application, skin cells.

Compositions can be administered once or multiple times over the course of a treatment period (e.g., until tolerance to an autoantigen is induced). In some instances, compositions are administered to the subject once per day, five times per week, four times per week, three times per week, twice per week, once every two weeks, once every three weeks, monthly, bimonthly, five times per year, four times per year, three times per year, twice per year, or once per year.

The examples that follow do not limit the scope of the embodiments described herein. One skilled in the art will appreciate that modifications can be made in the following examples which are intended to be encompassed by the spirit and scope of the invention.

### EXAMPLES

### Example 1

### In Vitro Degradation of ITE and its Inhibition

ITE is unstable in blood plasma and in tissue extracts, which may limit its efficacy. The methyl ester group of ITE is efficiently removed by an activity present in liver, plasma and skin. The metabolite is 2-(1H-Indol-3-ylcarbonyl)-4-thiazolecarboxylic acid (ITC). To determine whether hydrolysis of ITE was catalyzed by an esterase a panel of four esterase inhibitors (benzil, bis-para-nitrophenyl phosphate (BNPP), tacrine and rivastigmine) were evaluated for their ability to inhibit ITE degradation in mouse plasma. The properties of three inhibitors are summarized in the table below.

| | Esterase | | | |
|---|---|---|---|---|
| Inhibitor | CES1 (liver) | CES2 (gut) | AChE (blood) | BChE (blood) |
| Benzil | 0.37 ± 0.04 | 0.33 ± 0.03 | >100 | >100 |
| Tacrine | >100 | >100 | 0.079 ± 0.002 | 0.034 ± 0.001 |
| Rivastigmine | >100 | >100 | 299 ± 24.6 | 280 ± 110 |

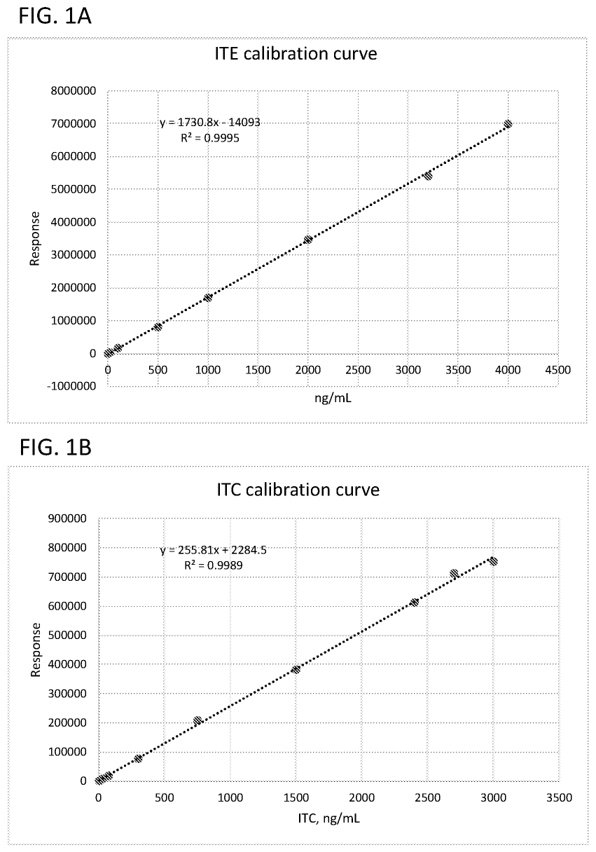

Levels of ITE and ITC in mouse plasma were determined by liquid chromatography-mass spectrometry (LC-MS). Standard curves were produced by serial dilution of ITE and ITE and used to convert ion counts to absolute concentrations (FIG. 1A).

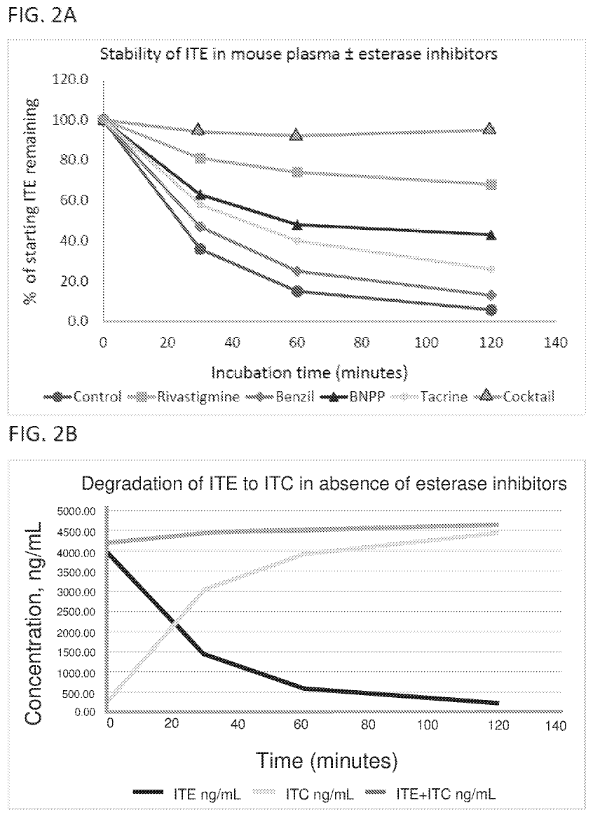

FIG. 2B shows the precursor-product relationship of ITE and ITC. When ITE was incubated in 0.5 milliliters of mouse plasma it is rapidly degraded (about 90% degraded by 60 minutes), while ITC accumulated at a commensurate rate. The top line shows the preservation of mass when ITE+ITC are summed. The flat line indicates that there were no other major metabolites of either ITE or ITC.

Each inhibitor was tested independently for its ability to protect ITE from degradation in 0.5 milliliters of mouse plasma (FIG. 2A). Inhibitors (1 millimolar benzil, BNPP and tacrine, 0.5 millimolar rivastigmine) were pre-incubated with plasma for 60 minutes before ITE was added at time=0. In the absence of any inhibitor, over 60% of ITE was lost at 30 minutes. Each inhibitor provided at least some protection from degradation. Rivastigmine was the best inhibitor of ITE degradation, protecting almost 70% of ITE from degradation at 120 minutes, followed by BNPP, tacrine, and benzil (FIG. 2A). When all four inhibitors were added together (“cocktail”) almost complete inhibition of ITE degradation was observed (over 90% of starting ITE was intact after two hours in mouse plasma).

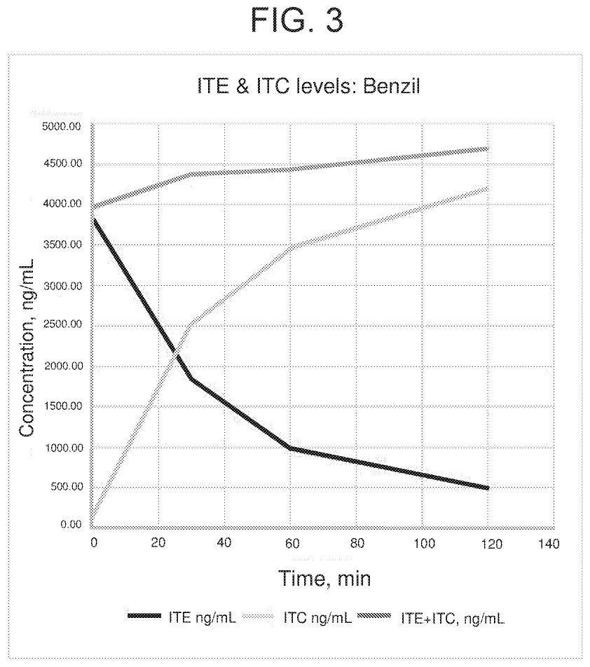

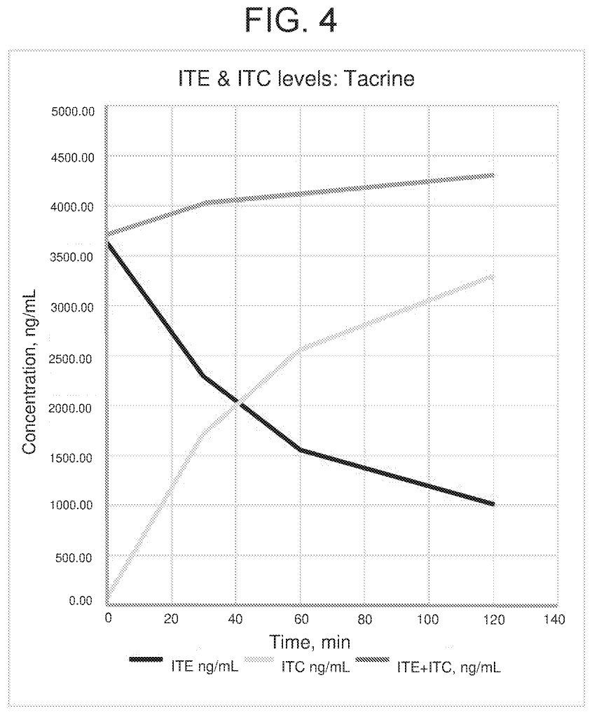

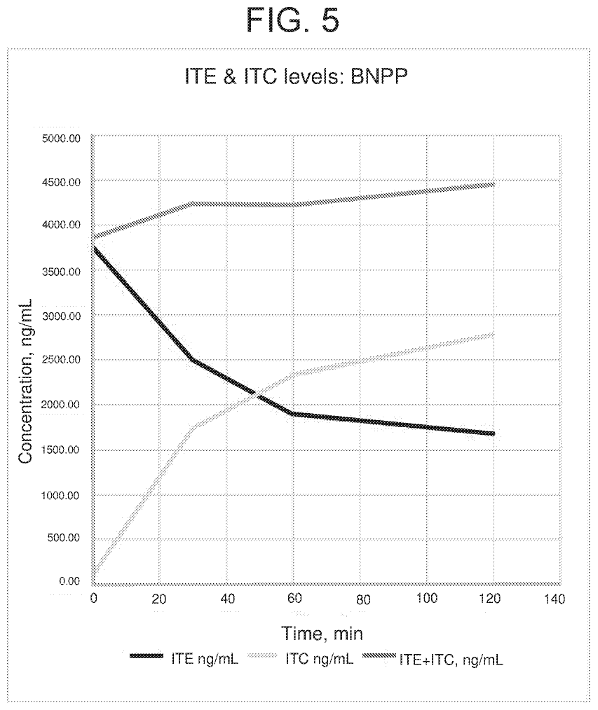

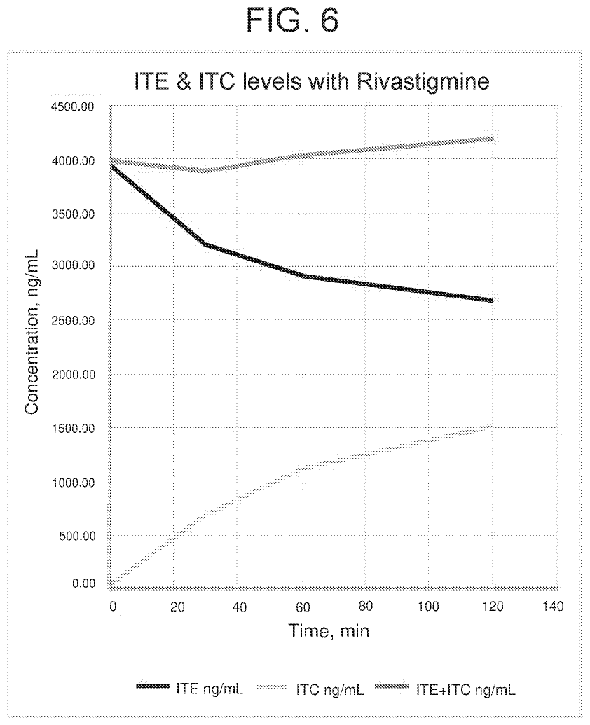

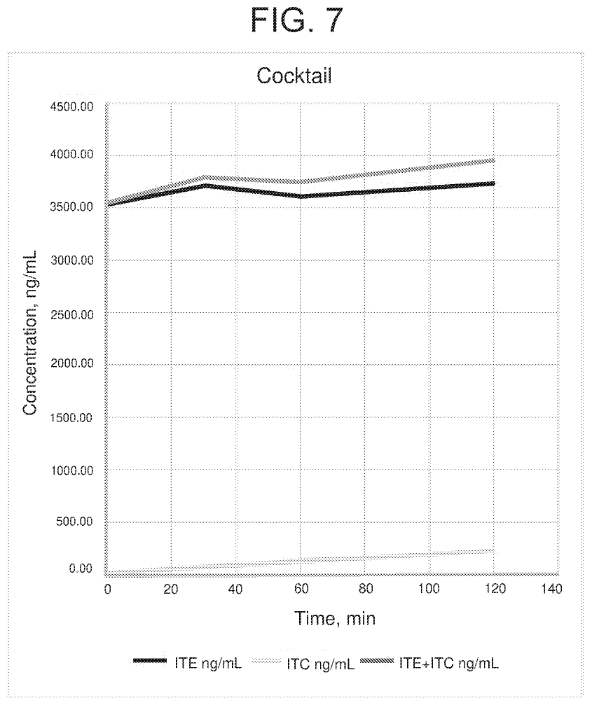

FIGS. 3-7 show a mass balance analysis of ITE+ITC in the presence of each inhibitor. The curves show the same pattern as FIG. 2A (cocktail >rivastigmine >BNPP >tacrine >benzil) and confirm that each inhibitor slows the rate of conversion of ITE to ITC. The effectiveness of the cocktail demonstrates that at least one of the three weaker inhibitors (BNPP, tacrine, benzil) inhibits an esterase not completely inhibited by rivastigmine. Benzil is a pan-carboxylesterase inhibitor (carboxylesterases 1 and 2), which appears to be most complementary to (least overlap with) rivastigmine, even though it is the weakest single inhibitor.

### Example 2

### Treatment of Psoriasis in a Mouse Model

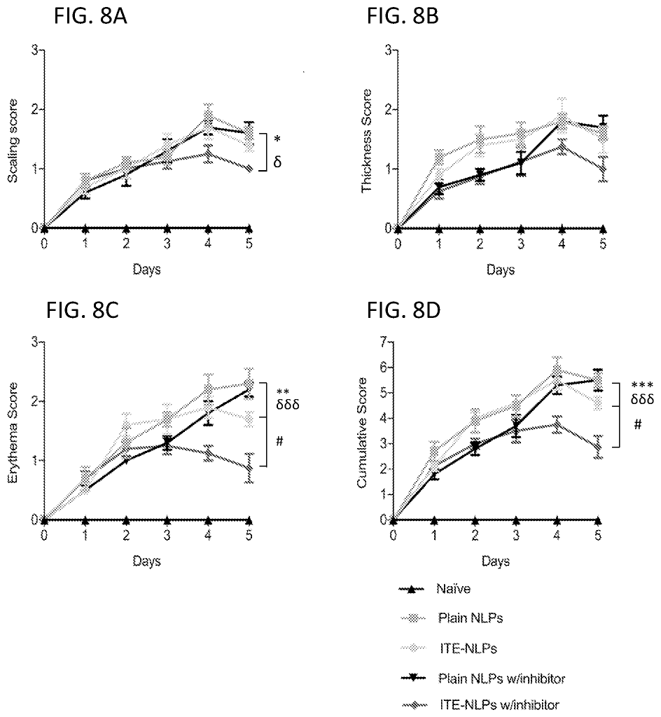

Psoriasis-like skin lesions were produced in rodents by painting the antiviral drug imiquimod on the shaved skin of the back. Both morphological and biochemical changes similar to those in human psoriasis appeared within a few days of applying imiquimod. Candidate treatments were evaluated objectively by having a trained, blinded rater score the skin lesions using the Psoriasis Area and Severity Index (PASI), the most widely used scale for measurement of the severity of psoriasis. The PASI score was decomposed into its components (scaling, skin thickness, and erythema; see FIGS. 8A, 8B and 8C), or combined in a composite score to produce an assessment of the overall severity of disease (FIG. 8D). Possible composite scores range from 0 (no disease) to 72 (maximal disease).

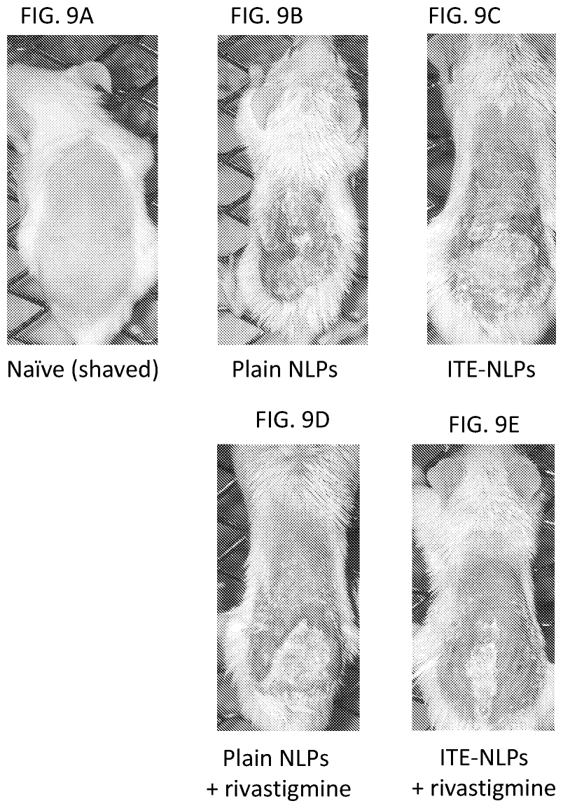

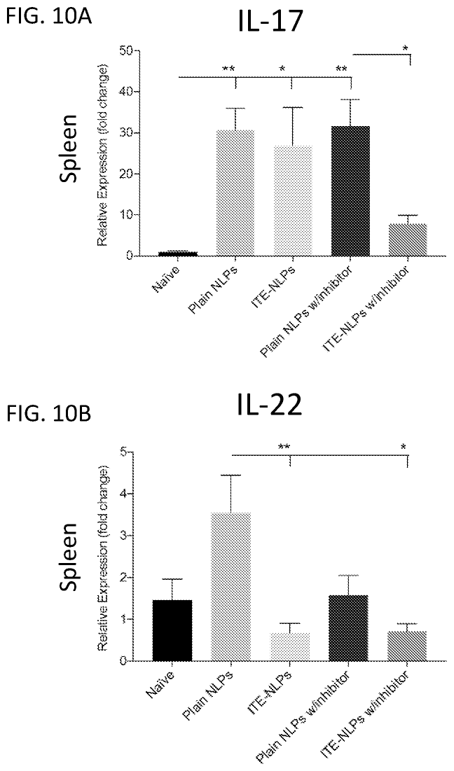

ITE was formulated in nanoparticulate liposomes and applied to the skin surface. Rivastigmine, an esterase inhibitor, was formulated for topical administration in a cream. Only when liposomes were co-administered with the esterase inhibitor was there maximal amelioration of psoriasis (FIGS. 8A-8D). Both skin lesions (FIG. 9E) and pro-inflammatory cytokines (FIGS. 10A and 10B) were reduced by the combination of ITE plus rivastigmine.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claims.

Other embodiments are within the claims.

What is claimed is:

1. A composition comprising 2-(1 H-Indol-3-ylcarbonyl)-4-thiazolecarboxylic acid methyl ester (ITE), or salt thereof, and an inhibitor of ITE degradation.

2. The composition of claim 1, wherein the composition comprises a population of nanoparticles, wherein the population of nanoparticles comprises the ITE, or salt thereof.

3. The composition of claim 1 or 2, wherein the inhibitor of ITE degradation is an esterase inhibitor.

4. The composition of claim 3, wherein the esterase inhibitor inhibits multiple esterases.

5. The composition of claim 4, wherein the esterase inhibitor inhibits a carboxylesterase, a plasma esterase, an acetylcholinesterase, a butyrylcholinesterase, and/or a paraoxonase.

6. The composition of any one of claims 2-5, wherein the esterase inhibitor is selected from the group consisting of rivastigmine, benzil, tacrine, and bis-para-nitrophenylphosphate (BNPP).

7. The composition of any one of claims 1-6, wherein the ITE and the inhibitor of ITE are co-formulated.

8. The composition of any one of claims 1-6, wherein the ITE and the inhibitor of ITE are separately formulated.

9. The composition of any one of claims 1-8, wherein the population of nanoparticles is a population of liposomes.

10. The composition of any one of claims 1-9, wherein the population of nanoparticles has an average diameter from 50-250 nanometers (nm).

11. The composition of any one of claims 1-10, wherein the population of nanoparticles is a population of liposomes having an average desaturation index of 0.4 or greater.

12. The composition of any one of claims 1-11, wherein the population of nanoparticles has an average from 200-15,000 molecules of 2-(1H-Indol-3-ylcarbonyl)-4-thiazolecarboxylic acid methyl ester (ITE), or salt thereof, per nanoparticle

13. The composition of any one of claims 1-12, wherein the population of nanoparticles is a population of liposomes comprising an average phase transition temperature from −60° C. to 80° C.

14. The composition of any one of claims 1-12, wherein the population of nanoparticles is a population of liposomes comprising a lipid mixture comprising a saturated lipid species and an unsaturated lipid species, wherein the unsaturated lipid species comprises an unsaturated bond and accounts for at least 20% of the lipid mixture.

15. The composition of claim 14, wherein the unsaturated lipid species comprises two lipid tails, wherein one or both lipid tails comprise a single unsaturated bond.

16. The composition of any one of claims 1-15, wherein the population of liposomes further comprises an antigen at a mass ratio of antigen-to-ITE from 1:10 to 10:1.

17. The composition of any one of claims 1-16, wherein the population of liposomes further comprises an antigen at a molar ratio of antigen-to-ITE from 1:100 to 10:1.

18. The composition of claim 16 or 17, wherein the antigen is a peptide antigen.

19. The composition of any one of claims 16-18, wherein the antigen is associated with an autoimmune disorder.

20. The composition of claim 19, wherein the autoimmune disorder is an autoimmune skin disease.

21. The composition of claim 20, wherein the autoimmune skin disease is selected from the group consisting of alopecia areata, bullous pemphigoid, dermatomyositis, epidermolysis bullosa, pemphigus vulgaris, psoriasis or scleroderma, vitiligo.

22. The composition of claim 21, wherein the autoimmune disorder is selected from the group consisting of rheumatoid arthritis, multiple sclerosis, type I diabetes, myasthenia gravis, inflammatory bowel disease, and celiac disease.

23. The composition of any one of claims 16-22, wherein the antigen is a therapeutic protein or a portion thereof.

24. The composition of any one of claims 1-23, wherein the population of liposomes has an average zeta potential from −10 and −50 mv.

25. The composition of any one of claims 16-24, wherein the antigen comprises a therapeutic agent.

26. The composition of claim 25, wherein the therapeutic agent is:
(i) a therapeutic protein or peptide;
(ii) a virus or capsid protein thereof; and/or
(iii) a polynucleotide encoding (i) and/or (ii).

27. The composition of claim 26, wherein the therapeutic agent is an adeno-associated virus (AAV), or capsid protein thereof.

28. The composition of claim 27, wherein the AAV comprises a DNA.

29. The composition of claim 28, wherein the DNA is a single stranded DNA (ssDNA).

30. The composition of claim 29, wherein the ssDNA encodes a cDNA or fragment thereof.

31. The composition of claim 30, wherein ssDNA encodes a human cDNA or fragment thereof.

32. The composition of claim 27, wherein the AAV comprises a RNA.

33. The composition of claim 32, wherein the RNA is a microRNA (miRNA).

34. A method of treating a subject having an autoimmune disorder, the method comprising administering to the subject the composition of any one of claims 1-33 in a therapeutically effective amount.

35. The method of claim 34, wherein the administration is an intravenous, subcutaneous, intradermal, dermal, intra-articular, pulmonary, or mucosal administration.

36. The method of claim 34 or 35, wherein a dose of the composition administered comprises from 1 μg to 50 mg ITE.

37. The method of claim 36, wherein a dose of the composition administered comprises from 100 μg to 1 mg ITE.

38. The method of any one of claims 34-37, wherein a dose of the composition administered comprises from 10 μg to 50 mg inhibitor of ITE degradation.

39. The method of claim 38, wherein a dose of the composition administered comprises from 1 mg to 20 mg inhibitor of ITE degradation.

40. The method of any one of claims 34-39, wherein a dose of the composition administered comprises from 1 μg to 50 mg ITE and from 10 μg to 50 mg inhibitor of ITE degradation.

41. A method of treating a subject having an autoimmune disorder, the method comprising administering to the subject (a) a population of nanoparticles comprising ITE, or salt thereof, and (b) an inhibitor of ITE degradation.

42. The method of claim 41, wherein the population of nanoparticles is administered through a separate route than the inhibitor of ITE degradation.

43. The method of claim 42, wherein the inhibitor of ITE degradation is administered with an antigen, wherein the antigen is associated with the autoimmune disorder.

44. The method of claim 42 or 43, wherein the inhibitor of ITE degradation is formulated in a gel, lotion, or cream for topical administration.

45. The method of any one of claims 34-44, wherein the autoimmune disorder is an autoimmune skin disease.

46. The method of claim 45, wherein the autoimmune skin disease is selected from the group consisting of psoriasis, alopecia areata, bullous pemphigoid, dermatomyositis, epidermolysis bullosa, pemphigus vulgaris, scleroderma, vitiligo.

47. A method of reducing the risk of an interfering immune response in a subject to be treated with a therapeutic agent, the method comprising administering to the subject the composition of any one of claims 25-33 in an amount sufficient to reduce immunogenicity to the therapeutic agent in the subject, thereby reducing the risk of an interfering immune response.
